(19) United States
(12) Patent Application Publication
(10) Pub. No.: US 2024/0316324 A1
(43) Pub. Date: Sep. 26, 2024

(54) DRUG DELIVERY DEVICE WITH RELEASABLE ATTACHMENT PART

(71) Applicant: Biograil APS, Roskilde (DK)

(72) Inventors: Nikolaj Skak, Roskilde (DK); Karsten Lindhardt, Roskilde (DK); Kamille Majken Dumong Erichsen, Roskilde (DK)

(21) Appl. No.: 18/260,865

(22) PCT Filed: Jan. 28, 2022
(86) PCT No.: PCT/EP2022/052054

§ 371 Date: Jul. 10, 2023

(30) Foreign Application Priority Data
Jan. 29, 2021 (DK) PA202170042
Oct. 28, 2021 (DK) PA202170527

(51) Int. Cl. A61M 31/00

(52) U.S. Cl. CPC A61M 31/002

(57) ABSTRACT

A drug delivery device having a central axis, the drug delivery device comprising a first body part, a second body part, an actuator mechanism configured to rotate at least one of the first body part or the second body part with respect to one another about the central axis, and a first attachment part comprising a connector component and a first spike component, the connector component connected to the first body part and the first spike component releasably connected to the connector component.

The present disclosure relates to a drug delivery device and in particular to a drug delivery device for oral administration. The drug delivery device is advantageously configured for delivery of an active drug substance in the gastrointestinal tract including the stomach and/or intestines, such as the small intestines and/or the large intestines (colon).

### BACKGROUND

A number of low permeable and/or low water soluble active drug substances are currently delivered by i.e. subcutaneous, intradermal, intramuscular, rectal, vaginal or intravenous route. Oral administration has the potential for the widest patient acceptance and thus attempts to deliver low permeable and/or low water soluble active drug substances through the preferred oral route of administration has been tried but with limited success in particular due to lack of stability and limited absorption from the gastrointestinal tract.

Stability both relates to the stability of the active drug substance during manufacturing and storage of the delivery device, and to the stability of the active drug substance during the passage in the gastrointestinal tract before it become available for absorption.

Limited gastrointestinal absorption is due to the gastrointestinal wall barrier preventing active drug substance from being absorbed after oral dosing because of the low permeability of the active drug substance, which is for example due to pre-systemic metabolism, size and/or the charges and/or because of the water solubility of the active drug substance.

Multiple approaches to solve these stability and absorption challenges have been suggested, but an effective solution to the challenges remain unresolved.

### SUMMARY

Thus, there is an unmet need to provide a drug delivery device, which is capable of delivering drug substances for absorption in the gastrointestinal tissue. More generally, there remains a need for drug products and methods that enable enhanced drug delivery, when drug products are administered orally to patients.

Disclosed herein is a drug delivery device having a central axis, the drug delivery device comprising a first body part, a second body part, an actuator mechanism configured to rotate at least one of the first body part or the second body part with respect to one another about the central axis, and a first attachment part. The first attachment part may comprise a connector component and a first spike component, the connector component optionally connected to the first body part. The first spike component may be releasably connected to the connector component.

A drug delivery device is disclosed, the drug delivery device comprising a first body part and a first attachment part comprising a connector component and a first spike component, the connector component connected to the first body part. The first spike component may be releasably connected to the connector component. Thus, a second body part and/or an actuator mechanism may be optional.

A drug delivery device is disclosed, the drug delivery device having a central axis. The drug delivery device comprises a first body part, a second body part, an actuator mechanism configured to rotate at least one of the first body part or the second body part with respect to one another about the central axis, and a first attachment part comprising a connector component and a first spike component. The connector component is connected to the first body part and the first spike component. The connector component or at least a section of the connector component is optionally configured to separate from the first body part.

It is an important advantage of a drug delivery device that the drug delivery device can be quickly released from a patient's system while still continuing to deliver an active drug substance to the patient, thereby reducing any bodily response to the drug delivery device. It is also an important advantage of the drug delivery system to be easy to assemble and/or manufacture. Additionally, it is an important advantage of the drug delivery device to provide improved delivery of an active drug substance.

The present disclosure allows for quick releasing of drug delivery devices from a patient while maintain dosing of an active drug substance. In particular, the drug delivery device may be used for attachment of the drug delivery device into a patient's system. A majority of the drug delivery device, e.g. the carrier, can then be quickly released via separation from a component of a first attachment part holding the active drug substance, while the active drug substance remains and continues to be provided to the patient. The first attachment part can then be later released and/or dissolved after providing the full does of active drug substance to the patient. As the first attachment part for the active drug substance can be significantly smaller than the whole drug delivery device, it can cause less of an adverse effect to the patient's system.

Further, the carrier can be optionally cleaned and/or sterilized and potentially reused with a new active drug substance in a new first attachment part. The updated drug delivery device with the new first attachment part can then be given back to the original patient for further use, or given to a new patient. This can further advantageously reduce waste as a significant portion of the drug delivery device can be reused.

Moreover, the present disclosure allows for improved assembly of drug delivery devices. In particular, the carrier and one or more components of the first attachment part can be manufactured separately, and then easily combined into the drug delivery device. Further, either the carrier or the first attachment part can be modified as required, allowing for a modular approach to a drug delivery system. Thus, the first attachment part can be used with multiple types of carriers, and vice versa.

### BRIEF DESCRIPTION OF THE DRAWINGS

The above and other features and advantages of the present invention will become readily apparent to those skilled in the art by the following detailed description of exemplary embodiments thereof with reference to the attached drawings, in which:

FIG. 1 shows a perspective view of an exemplary drug delivery device,

FIGS. 2-3 show an exploded view of an exemplary drug delivery device,

FIG. 4 shows a perspective view of a first body part and a second body part of an exemplary drug delivery device,

FIG. 5 shows an exemplary attachment part, such as a first attachment part,

FIG. 6 shows a perspective view of an exemplary drug delivery device releasing an attachment part, such as a first attachment part,

FIG. 7 shows an alternate exemplary attachment part, such as a first attachment part,

FIG. 8 shows an exemplary attachment part, such as a first attachment part, without a coupling component,

FIG. 9 shows an exemplary attachment part, such as a first attachment part, with a translating coupling component,

FIG. 10 shows an alternate exemplary attachment part, such as a first attachment part, with a mating protrusion,

FIG. 11 shows an exemplary drug delivery device having a separatable connector component,

FIG. 12 shows an exemplary drug delivery device after separation of the connector component, and

FIGS. 13A-13C show an exemplary drug delivery device with a releasable attachment part.

### DETAILED DESCRIPTION

Various exemplary embodiments and details are described hereinafter, with reference to the figures when relevant. It should be noted that the figures may or may not be drawn to scale and that elements of similar structures or functions are represented by like reference numerals throughout the figures. It should also be noted that the figures are only intended to facilitate the description of the embodiments and the functionalities associated therewith. They are not intended as an exhaustive description of the invention or as a limitation on the scope of the invention or the physical appearance of the invention. In addition, an illustrated embodiment needs not have all the aspects or advantages shown. An aspect or an advantage described in conjunction with a particular embodiment is not necessarily limited to that embodiment and can be practiced in any other embodiments even if not so illustrated, or if not so explicitly described.

A drug delivery device having a central axis is disclosed, the drug delivery device comprising a first body part, a second body part, an actuator mechanism configured to rotate at least one of the first body part or the second body part with respect to one another about the central axis, and a first attachment part comprising a connector component and a first spike component, the connector component connected to the first body part and the first spike component releasably connected to the connector component.

A drug delivery device is disclosed. In one or more exemplary drug devices, drug delivery device has having central axis. The drug delivery device includes a first body part. In one or more exemplary drug devices, drug delivery device includes a second body part. In one or more exemplary drug devices, drug delivery device includes an actuator mechanism configured to rotate at least one of the first body part or the second body part with respect to one another about the central axis. In one or more exemplary drug devices, drug delivery device includes a first attachment part connected to the first body part. In one or more exemplary drug devices, the first attachment part is configured to separate from the first body part.

In one or more exemplary drug delivery devices, the drug delivery device may be a carrier in combination with a first attachment part, e.g. delivery part, attachment part, or a component of a first attachment part. The first attachment part and/or the component of the first attachment part, such as a first spike component, may be associated with a carrier. For example, the first attachment part and/or the component of the first attachment part may be releasably attached to the carrier. In one or more exemplary drug delivery devices, the drug delivery device may be a carrier. Accordingly, discussion with respect to the drug delivery device may be applicable to the carrier, and vice versa.

The drug delivery device may have a size and geometry designed to fit into a pharmaceutical composition for oral administration.

The drug delivery device/pharmaceutical composition may be configured to be taken into the body via the oral orifice. Thus, the outer dimensions of the drug delivery device/pharmaceutical composition may be small enough for a user to swallow. The drug delivery device may be adapted to carry a drug substance (e.g., an active drug substance) into the body of the user, via the digestive system, so that the drug delivery device may e.g., travel from the mouth of the user into the stomach, via the oesophagus. The drug delivery device may further travel into the intestines from the stomach, and may optionally travel into the bowels and out through the rectum.

The drug delivery device may be configured to deliver the drug in any part of the digestive system of the user, where in one example it may be configured to deliver a drug substance into the stomach of the user. In another example, the drug delivery device may be adapted to initiate the drug delivery when the device has passed the stomach and has entered the intestine of the user. In other words, the drug delivery device may be configured to attach to a wall of the stomach or a wall of the intestines, e.g., depending on the desired release position of the active drug substance.

The attachment part(s) of the drug delivery device may be configured to interact with the inner surface linings of the gastrointestinal tract, so that the drug delivery device may e.g., be attached to the inner surface (mucous membrane) of the stomach, or alternatively to the mucous membrane of the intestines. The attachment part(s), e.g. first attachment part and/or second attachment part and/or third attachment part, may be configured to interact with the mucous membranes, e.g., in order to fix or attach the drug delivery device, e.g., for a period of time, inside the body of the user. By attaching the drug delivery device, it will allow a drug substance to be delivered into a part of the digestive system, in order to provide a drug substance to the body of the user. The attachment part(s) may be configured to interact with the mucous membranes, e.g., in order to inject drug substance into the gastrointestinal tract wall. In one or more exemplary drug delivery devices, the attachment part(s) may remain in the linings to provide the drug substance while a remainder of the drug delivery device is released, such as separated and/or detached. The attachment part(s) may be considered as delivery part(s).

The drug delivery device has a central axis optionally extending from a first end to a second end of the drug delivery device. The drug delivery device may have a length (e.g. largest extension from first end to second end along central axis), in the range from 3 mm to 35 mm, such as in the range from 5 mm to 26 mm. The drug delivery device may be elongated.

The drug delivery device may have a width and/or height (e.g. largest extensions along width axis and height axis, respectively) in the range from 1 mm to 20 mm. Height and width are the largest extensions of the drug delivery device perpendicular to the central axis.

In one or more exemplary drug delivery devices, the dimensions of the drug delivery device, at least in an initial state or first state prior to actuation of the first attachment part and/or the second attachment part, may be represented by a length (largest extension along central axis), a width (largest extension along width axis perpendicular to the central axis) and a height (largest extension along height axis perpendicular to the central axis and the width axis). The height of the drug delivery device may be in the range from 1 mm to 15 mm. The width of the drug delivery device may be in the range from 1 mm to 15 mm.

In one or more exemplary drug delivery devices, the drug delivery device may be constructed in a way that secures the drug delivery part to deliver a payload or active drug substance into the internal tissue or internal surface for distribution of the active drug substance in the subject through the blood vessels.

Advantageously, the drug delivery device may be attached, and may deliver the active drug substance, to a particular location in a patient's intestinal wall. Of course, the delivery device may be attached, and may deliver the active drug substance, to other places as well. In one or more exemplary drug delivery devices, the drug delivery device, such as the spike, may penetrate the muscularis mucosa. In one or more exemplary drug delivery devices, the drug delivery device, such as the spike, may not penetrate the muscularis externa. In one or more exemplary drug delivery devices, the spike may be positioned in the submucosa. In one or more exemplary drug delivery devices, the spike may be positioned in the submucosa parallel to the gut wall.

The drug delivery device includes a first body part. The first body part may be a one-part body part, e.g., unitary. The first body part may be a two-part body part, i.e. the first body part may comprise a first primary body part and a first secondary body part. The first body part has an outer surface. A first recess may be formed in the outer surface of the first body part. Alternatively, a first primary recess and/or a first secondary recess may be formed in the outer surface of the first body part.

The drug delivery device includes a first attachment part. The first attachment part may comprise a first base part and/or a first spike component, e.g. spike component, needle, spike, spear. The first attachment part has a first proximal end, e.g. proximal end, and a first distal end, e.g. distal end. The first attachment part, such as the first spike component, optionally has or extends along a first attachment axis. A first tip of the first spike component forms the first distal end. In other words, the first distal end is a first tip of the first spike component. The first base may be arranged at or constitute the first proximal end of the first attachment part. The first spike component may have a length in the range from 1 mm to 15 mm such, as in the range from 3 mm to 10 mm. Thereby sufficient penetration into the internal tissue may be provided for while at the same time reducing the risk of damaging the internal tissue. The first distal end of the first attachment part may be provided with a tip configured to penetrate a biological tissue. The first distal end of the first attachment part may be provided with a gripping part configured to grip a biological tissue.

The first spike component may have a cross-sectional diameter in the range from 0.1 mm to 5 mm, such as in the range from 0.5 mm to 2.0 mm.

The first spike component may be straight and/or curved. The first spike component may comprise a first primary section that is straight. The first spike component may comprise a first secondary section, e.g. between the first primary section and the first distal end or between the first base and the first primary section. The first secondary section may be curved.

The first spike component may include two or more straight portions formed at an angle. For example, the first spike component may have a proximal portion that extends at a first angle from a connection point to the drug delivery device and a distal portion that extends at a second angle from a connection point to the drug delivery device. The first angle and the second angle may be different. The proximal portion may connect to the distal portion at a joint (e.g., bend, connection, angle) and have a joint angle between the proximal portion and the distal portion. The joint angle may be an acute angle, an obtuse angle, or a right angle. The angle may be, for example, 10, 20, 30, 40, 50, 60, 70, 80, 90, 100, 120, 130 140, 150, 160, or 170 degrees. This can advantageously allow for different angles of attach when the first spike component interacts with inner surface linings. This may allow for improved attachment of the drug delivery device, while helping to reduce or avoid tissue damage. Further, the joint may be flexible. Alternatively, the joint may not be flexible.

The joint may be located at a center, or generally at a center, of a length of the first spike component. Alternatively, the joint may be located 40, 45, 55, 60, or 65% up a length of the first spike component from the proximal end.

In one or more exemplary drug delivery devices, the first spike component may have three, four, or five different portions at different angles, each connected by a joint. In some iterations, any or all of the different portions may be straight or curved. Each joint may be flexible or not flexible.

The attachment parts (e.g., the first attachment part and/or the second attachment part discussed below) of the carrier and/or drug delivery device may be seen as any kind of attachment parts that may be capable of attaching the drug delivery device to a biological tissue, such as a stomach wall, a wall of the bowels and/or intestines of a human or animal body. The attachment parts may be adapted to extend in a direction away from the central axis of the drug delivery device and/or carrier, and/or a central axis of the first attachment part. This may mean that the attachment part(s), e.g., at least in an activated state or second state of the drug delivery device, may extend in a direction away from a peripheral surface (in radial direction) of the first body part and/or the second body part, so that the attachment part extends farther in a radial direction than the peripheral or outer surface of the body part.

The first attachment part may be fixed or rotationally attached to the first body part. In particular, the first attachment part may be rotationally attached within the first recess, the first recess being discussed in detail below.

An attachment part, such as the first attachment part and/or the second attachment part, may be made up of one or more components or pieces. An attachment part, such as the first attachment part and/or the second attachment part), can include some, any, and/or all of the components discussed herein with respect to the attachment part.

For example, a first attachment part can include a connector component of the first attachment part and/or a coupling component of the first attachment part and/or a hinge of the first attachment part and/or a first spike. For example, a second attachment part can include a connector component of the second attachment part and/or a coupling component of the second attachment part and/or a hinge of the second attachment part and/or a second spike.

In one or more example drug delivery devices, one or more components of the attachment part, such as the first attachment part and/or the second attachment part, can be releasable from the drug delivery device, such as from the carrier and/or the first body part and/or the second body part. In one or more example drug delivery devices, one or more components of the attachment part, such as the first attachment part and/or the second attachment part, can be separable from the drug delivery device, such as from the carrier and/or the first body part and/or the second body part. Releasable and/or separable can also be, for example, one or more of detachable, removable, degradable, and breakable.

One or more components of the attachment part, such as the first attachment part and/or the second attachment part, can be considered releasable, such as separable, if the one or more components of the attachment part, such as the first attachment part and/or the second attachment part, degrades, either partially or fully. For example, if an entirety of the attachment part, such as the first attachment part and/or the second attachment part, degrades and/or dissolves, it can be considered as released or separated.

For example, the attachment part, such as the first attachment part and/or the second attachment part, can be released from the drug delivery device, such as from the carrier and/or the first body part and/or the second body part, so that the remainder of the drug delivery device, such as from the carrier and/or the first body part and/or the second body part, can pass through a patient's body while the attachment part, such as the first attachment part and/or the second attachment part, remains in the patient's body at least for a certain time period necessary for delivery of the active drug substance.

The attachment part, such as the first attachment part and/or the second attachment part, may be made from a dissolvable material. The attachment part, such as the first attachment part and/or the second attachment part, may be made from a degradable material. The attachment part, such as the first attachment part and/or the second attachment part, may be made from a biodegradable material. For example, the attachment part, such as the first attachment part and/or the second attachment part, may be made partially or fully out of MgZnCa or an alloy comprising one or more of Mg, Zn, and Ca. A portion, such as a section or component, of the attachment part, such as the first attachment part and/or the second attachment part, may be made partially or fully out of MgZnCa or an alloy comprising one or more of Mg, Zn, and Ca. For example, one or more of the first spike, coupling component, and connector component can be made partially or fully out of MgZnCa or an alloy comprising one or more of Mg, Zn, and Ca.

In one or more exemplary drug delivery devices, the first attachment part may include one or more components, e.g. parts, intermediates, pieces. One or more of the one or more components may be releasably connected to the drug delivery device, such as the first body part.

In one or more exemplary drug delivery devices, the first attachment part may include a connector component, e.g. arm, holder, branch, member. A first spike component, e.g. needle, spear, spike, can be located and/or associated at or on a distal end of the connector component. The connector component may be straight. The connector component may be bent. The connector component may include a number of bends. The connector component may be curved. The connector component can be a single unit. The connector component can be a number of segments attached together. The number of segments may be rigidly attached together. The number of segments may be rotatably attached.

The connector component can be rotatably held within the first recess. Thus, the connector component of the first attachment part can be rotatably connected to the first body part. In a first position, the connector component can be held fully within the first recess. In a second position, the connector component may at least partially extend out of the first recess. Thus, a distal end of the connector component can rotate from within the recess to outside of the recess. This movement can occur during rotation of the first body part.

In the second position, the connector component may abut a surface of the first recess. This can prevent further motion of the connector component. Alternatively, other stopping mechanisms can be used to prevent further motion of the connector component.

Further, other positions, such as a third, fourth, and/or fifth position can be used. These further positions can be between the first and second position or outside the first and second positions.

Thus, the first spike component can rotate with respect to the connector component, such as on an axis at the distal end of the first arm. The first spike component may not rotate with respect to the connector component. A proximal end of the connector component can be associated with the first attachment part axis, discussed below. Thus, the connector component and first spike component can rotate with respect to the first attachment part axis.

While rotational motion is discussed herein, it will be understood that other translations and/or motions can be used as well. For example, straight, longitudinal, radial, and circumferential motion can all be used to translate the connector component between the first position and the second position.

The connector component can be shaped to fit within the first recess. For example, if the first recess has a rectangular cross section, the connector component may have a rectangular cross section as well. However, the particular shape of the connector component is not limiting. The connector component may have a square, oval, circle, triangle, and/or polygon cross section.

In one or more exemplary drug delivery devices, a distal end of the connector component may include a seat, e.g. cradle, mating surface. The seat may be configured to connect with, e.g. associate with, mate with, contact, a coupling component. For example, the seat may be configured to accept a cylindrical coupling component. The seat may have mating features to connect with the coupling component. Alternatively, the distal end of the connector component may not have a particular seat. The connector component may be chemically and/or mechanically attached to the coupling component. The coupling component may be releasably connected to the connector component. The coupling component may be permanently connected to the connector component.

In one or more exemplary drug delivery devices, a proximal end of the connector component (or the proximal end of the attachment part) may include an attachment point, e.g., attachment surface, attachment mechanism, attachment plane, attachment section, attachment component, attachment structure, attachment, connection, rotatable connection, to the respective body part. For example, the first attachment part, such as a connector component of the first attachment part may include an attachment point, such as a first attachment point with the first body part and the second attachment part, such as a second connector component of the second attachment part) may include an attachment point, such as a second attachment point with the second body part.

In one or more exemplary drug delivery devices, the connector component may include a dissolvable component, such as a dissolvable plug. The dissolvable component may be dissolvable, degradable, and/or biodegradable. The dissolvable component may be considered as part of the connector component and/or part of the attachment part, such as the first attachment part and/or the second attachment part. The dissolvable component may be a component capable of weakening. The dissolvable component may only weaken and not fully dissolve. The dissolvable component may weaken upon contact with a fluid.

The dissolvable component may be configured to prevent separation of the connector component from the first body part and/or the second body part. For example, the dissolvable component may be configured to prevent separation of the attachment part, such as the first attachment part and/or the second attachment part or part thereof from the first body part and/or the second body part.

For example, the dissolvable component may be configured to prevent translational movement of the attachment part, such as the first attachment part and/or the second attachment part, away from the first body part and/or the second body part, such as radially away. The dissolvable component may still allow for rotational movement of the attachment part, such as the first attachment part and/or the second attachment part, with respect to the first body part and/or the second body part.

Dissolvable of the dissolvable component may allow the attachment part, such as the first attachment part and/or the second attachment part, to translate away from the first body part and/or the second body part, e.g., separating the attachment part, such as the first attachment part and/or the second attachment part, from the first body part and/or the second body part.

For example, the dissolvable component may be located in a slot, such as a gap and/or pathway, in or of the attachment part, such as the first attachment part and/or the second attachment part, such as in the connector component. The dissolvable component can stop, such as block, movement through the slot. Upon dissolving of the dissolvable component, the slot becomes open, such as unblocked. The unblocking of the slot can allow the attachment part, such as the first attachment part and/or the second attachment part), such as in the connector component, to translate away from the drug delivery device, such as the first body part and/or the second body part. The translating away may occur to forces acting on the drug delivery device. The translating away may occur due to the forces applied by the actuator mechanism.

For example, the drug delivery device, such as the first body part and/or the second body part, may include a slot, such as a gap and/or pathway. The dissolvable component can be located in the slot. Upon dissolution of the dissolvable component, the attachment part, such as the first attachment part and/or the second attachment part, such as in the connector component, can pass through the slot to translate away from the drug delivery device, such as the carrier, such as the first body part and/or the second body part.

The dissolvable component may act as a back stop, such as preventing further rotation of the attachment part, such as the first attachment part and/or the second attachment part. Upon dissolving of the dissolvable component, the attachment part, such as the first attachment part and/or the second attachment part, can continue to rotate in the same direction as previously rotating. For example, the actuator mechanism may continue providing a force to rotate the first body part and/or the second body part. For example, the distal tips of the first attachment part and/or second attachment part can continue separating apart.

The continued rotation may be configured to allow the attachment part, such as the first attachment part and/or the second attachment part, to pass through a slot in the drug delivery device, such as the first body part and/or the carrier. For example, the attachment part, such as the first attachment part and/or the second attachment part, may include a protrusion preventing the attachment part, such as the first attachment part and/or the second attachment part, from passing through the slot. The protrusion may be shaped to pass through the slot in a particular orientation.

Continued rotation of the attachment part, such as the first attachment part and/or the second attachment part, may allow for the protrusion to be aligned with the slot to allow the attachment part, such as the first attachment part and/or the second attachment part, to pass through the slot, for example allowing for translation of the attachment part, such as the first attachment part and/or the second attachment part, away from the drug delivery device.

In one or more exemplary drug delivery devices, the dissolvable component may be part of the drug delivery device. In one or more exemplary drug delivery devices, the dissolvable component may be part of the drug delivery device. For example, the dissolvable component may be part of the first body part and/or the second body part. The dissolvable component can include any and/or all of the features discussed above.

In one or more example drug delivery devices, the first body part and/or the first attachment part includes a dissolvable component configured to prevent separation of the first attachment part away from the first body part. In one or more example drug delivery devices, the dissolvable component is located in a slot in the first body part or the first attachment part. In one or more example drug delivery devices, the first body part includes the dissolvable component. In one or more exemplary drug delivery devices, the first attachment part includes the dissolvable component. In one or more exemplary drug delivery devices, the dissolvable component connects is an attachment point between the first attachment part and the first body part. In one or more exemplary drug delivery devices, the attachment part is formed from a biodegradable material. In one or more exemplary drug delivery devices, the biodegradable material is MgZnCa.

The attachment point may include the attachment part, such as the first attachment part and/or the second attachment part. The attachment point may include the body part, such as the first body part and/or the second body part. The attachment point may include both the attachment part, such as the first attachment part and/or the second attachment part, and the body part, such as the first body part and/or the second body part. Both the attachment part, such as the first attachment part and/or the second attachment part, and the body part, such as the first body part and/or the second body part, may attach to or at the attachment point.

In one or more exemplary drug delivery devices, the first attachment part, such as a connector component of the first attachment part, can be configured to rotate at the first attachment point with respect to the first body part and the second attachment part, such as a connector component of the second attachment part, can be configured to rotate at the second attachment point with respect to the second body part.

The attachment point, such as the first attachment point and/or the second attachment point, may be, for example a hinge. The attachment point, such as the first attachment point and/or the second attachment point, may be for example a ball & socket The attachment point, such as the first attachment point and/or the second attachment point, may be, for example a pin and connector surrounding the pin. The attachment point, such as the first attachment point and/or the second attachment point, may be, for example a joint. The type of attachment point is not limiting.

The attachment point, such as the hinge, may be part of the attachment part, such as the first attachment part and/or the second attachment part. The attachment point, such as the hinge, may be part of the body part, such as the first body part and/or the second body part). The attachment point may be part of the drug delivery device. The attachment point, such as the hinge, may be a combination of components of the drug delivery system, such as a combination of the first attachment part and the first body part and/or a combination of the second attachment part and the second body part.

The attachment point, such as the hinge, may be made from a dissolvable material. The attachment point, such as the hinge, may be made from a degradable material. The attachment point, such as the hinge, may be made from a biodegradable material. For example, the attachment point, such as the hinge, may be made partially or fully out of MgZnCa or an alloy comprising one or more of Mg, Zn, and Ca. A portion, such as a section or component, of the attachment point, such as the hinge, may be made partially or fully out of MgZnCa or an alloy comprising one or more of Mg, Zn, and Ca. The attachment part may be formed of a dissolvable material. The attachment part may be formed partially or fully out of MgZnCa or an alloy comprising one or more of Mg, Zn, and Ca.

The dissolvable material may be dissolvable, degradable, and/or biodegradable. The dissolvable material may be a material capable of weakening. The dissolvable material may only weaken and not fully dissolve. The dissolvable material may weaken upon contact with a fluid.

The coupling component may be configured to retain, e.g. hold, contain, surround, the first spike component, e.g. spike, needle, spear. For example, the coupling component can be or comprise a sleeve, e.g. tube, cylinder, tunnel, conduit, configured to at least partially or fully surround a portion of the first spike component. In one or more exemplary drug delivery systems, the coupling component can be a sleeve, e.g. a cylinder, which can receive the first spike component within a lumen of the coupling component. Thus, the coupling component can connect or associate the first spike component with the connector component.

As mentioned, the coupling component may be a sleeve configured to surround a portion of the first spike component. The sleeve may form a fluid-tight connection or a semi-fluid tight connection with the first spike component, as discussed below. Alternatively, the coupling component may be a rail and/or platform and/or cradle and/or holder for the first spike component to associate with, releasably or permanently, so that the first spike component is associated with the connector component.

The connector component may include one or more mating features for connecting to the coupling component. For example, the connector component may include a mating protrusion which is accepted by a mating receiver on the coupling component, or vice versa.

In one or more exemplary drug delivery devices, the sleeve can be configured to at least partially surround the connector component. This can attach the sleeve and/or the coupling component to the connector component.

The sleeve can be formed by one or more different materials. For example, the sleeve may be water insoluble. Accordingly, the sleeve may be made from one or more of polyethylene, polypropylene, and acrylonitrile butadiene styrene. The sleeve may be water soluble. Accordingly, the sleeve may be made from one or more of polyethylene oxide and polyethylene glycol. The sleeve may be pH dependent for dissolving. Accordingly, the sleeve may be made from one or more of poly(methyl methacrylate), hydroxypropylmethylcellulose acetate succinate, and hypromellose phthalate.

In one or more exemplary drug delivery systems, the sleeve may have a longitudinal length larger than 1 mm, such as in the range from 1.5 mm to 15.0 mm, e.g. of 1.5, 1.6, 1.7, 1.8, 1.9, 2.0, 2.1, 2.1, 2.2, 2.3, 2.4, 2.5, 2.6, 2.7, 2.8, 2.9, 3.0, 3.1, 3.2, 3.3, 3.4, 3.5, 3.6, 3.7, 3.8, 3.9, 4.0, 4.1, 4.2, 4.3, 4.4, 4.5, 4.6, 4.7, 4.8, 4.9, 5.0, 6, 7, 8, 9, 10, 11, 12, 13, 14, or 15 mm. In one or more exemplary drug delivery systems, the sleeve may have a longitudinal length of greater than 1.5, 1.6, 1.7, 1.8, 1.9, 2.0, 2.1, 2.1, 2.2, 2.3, 2.4, 2.5, 2.6, 2.7, 2.8, 2.9, 3.0, 3.1, 3.2, 3.3, 3.4, 3.5, 3.6, 3.7, 3.8, 3.9, 4.0, 4.1, 4.2, 4.3, 4.4, 4.5, 4.6, 4.7, 4.8, 4.9, 5.0, 6, 7, 8, 9, 10, 11, 12, 13, 14, or 15 mm. In one or more exemplary drug delivery systems, the sleeve may have a longitudinal length of less than 1.5, 1.6, 1.7, 1.8, 1.9, 2.0, 2.1, 2.1, 2.2, 2.3, 2.4, 2.5, 2.6, 2.7, 2.8, 2.9, 3.0, 3.1, 3.2, 3.3, 3.4, 3.5, 3.6, 3.7, 3.8, 3.9, 4.0, 4.1, 4.2, 4.3, 4.4, 4.5, 4.6, 4.7, 4.8, 4.9, 5.0, 6, 7, 8, 9, 10, 11, 12, 13, 14, or 15 mm.

In one or more exemplary drug delivery systems, the sleeve may have a diameter, such as outer diameter, larger than 0.1 mm, such as in the range from 0.15 to 2 mm, e.g. of 0.1, 0.2, 0.3, 0.4, 0.5, 0.6, 0.7, 0.8, 0.9, 1.0, 1.2, 1.3, 1.4, 1.5, or 1.6, 1.7, 1.8, 1.9, or 2 mm. In one or more exemplary drug delivery systems, the sleeve may have a diameter of greater than 0.1, 0.2, 0.3, 0.4, 0.5, 0.6, 0.7, 0.8, 0.9, 1.0, 1.2, 1.3, 1.4, 1.5, or 1.6, 1.7, 1.8, 1.9, or 2 mm. In one or more exemplary drug delivery systems, the sleeve may have a diameter of less than 2.0 mm, such as less than 0.1, 0.2, 0.3, 0.4, 0.5, 0.6, 0.7, 0.8, 0.9, 1.0, 1.2, 1.3, 1.4, 1.5, or 1.6, 1.7, 1.8, 1.9, or 2 mm. In one or more exemplary drug delivery systems, the sleeve may have a diameter, such as outer diameter, larger than 0.1 mm, such as in the range from 0.15 to 2.5 mm

In one or more exemplary drug delivery systems, the sleeve may have a wall thickness of 0.5, 0.6, 0.7, 0.8, 0.9, 1.0, 1.2, 1.3, 1.4, or 1.5 mm. In one or more exemplary drug delivery systems, the sleeve may have a wall thickness of greater than 0.5, 0.6, 0.7, 0.8, 0.9, 1.0, 1.2, 1.3, 1.4, or 1.5 mm. In one or more exemplary drug delivery systems, the sleeve may have a wall thickness of less than 0.5, 0.6, 0.7, 0.8, 0.9, 1.0, 1.2, 1.3, 1.4, or 1.5 mm.

In one or more exemplary drug delivery systems, the sleeve may be configured to cover 10, 20, 30, 40, 50, 60, 70, 80, 90, or 100% of a longitudinal length of the first attachment part. In one or more exemplary drug delivery systems, the sleeve may be configured to cover greater than 10, 20, 30, 40, 50, 60, 70, 80, or 90% of a longitudinal length of the first attachment part. In one or more exemplary drug delivery systems, the sleeve may be configured to cover less than 10, 20, 30, 40, 50, 60, 70, 80, 90, or 100% of a longitudinal length of the first attachment part.

Thus, in one or more exemplary drug delivery devices, the first attachment part can include a coupling component. The coupling component can couple the first spike component to the connector component. The coupling component can be configured to decouple, e.g. release, remove, from the first spike component. When the coupling component decouples, the first spike component is released from the connector component. There are a number of options for the decoupling discussed below, each of which can be used alone or in combination.

In one or more exemplary drug delivery devices, the coupling component is made of a dissolvable material, and thus the coupling component is dissolvable. In one or more exemplary drug delivery devices, the coupling component is not made of a dissolvable material. In one or more exemplary drug delivery devices, the first spike component is made of a dissolvable material. In one or more exemplary drug delivery devices, the first spike component is not made of a dissolvable material.

In one or more exemplary drug delivery devices, the first spike component includes a chamber, e.g. compartment. The chamber can be configured to hold, for example, an active drug substance. The drug delivery device can be configured to deliver an active drug substance from the chamber to the surroundings of the drug delivery device. In one or more exemplary drug delivery devices, the first spike component comprises a chamber, the drug delivery device being configured to deliver an active drug substance from the chamber to the surroundings of the drug delivery device.

The first spike component can include multiple chambers. Each chamber may hold a same active drug substance, or a different active drug substance. The chambers may be spaced circumferentially or longitudinally around the first spike component.

The chamber may be arranged in the first attachment part, such as in the first spike component, e.g., within a distance of 8 mm, such as within 5 mm, from the first distal end. The first attachment part, such as the first spike component, may have one or more openings providing access to the chamber. In one or more exemplary drug delivery devices, the chamber is formed as a through-going bore in the first spike component.

In one or more exemplary drug delivery devices, the chamber may be open from an inner volume of the drug delivery device and towards an outer part of the drug delivery device. In one or more examples, the chamber may be inside the first spike component so that when the first distal end of the first attachment part has penetrated the biological tissue, the drug substance may be released from the chamber and into the biological tissue via the first spike component. This may e.g., be where the first spike component is a tubular part, which has a first distal end in fluid communication with the chamber.

In one or more exemplary drug delivery devices, the drug delivery device comprises a second chamber, the drug delivery device being configured to deliver an active drug substance from the second chamber to the surroundings of the drug delivery device. The second chamber may be arranged in the second attachment part, such as in the second spike component, e.g., within a distance of 8 mm, such as within 5 mm, from the second distal end. The second attachment part, such as the second spike component may have one or more openings providing access to the second chamber. In one or more exemplary drug delivery devices, the second chamber is formed as a through-going bore in the first spike component or in the second spike component.

The chamber can be exposed to an outer surface of the first spike component. This can allow the active drug substance to be released from the first spike component, such as into a patient. In order to prevent the active drug substance from being released too early, the coupling component, e.g. sleeve, can surround, e.g. cover, the chamber. Specifically, the sleeve can be in fluid-tight or semi-fluid tight connection with the first spike component, specifically over the chamber, to prevent release of the active drug substance.

In one or more exemplary drug delivery devices, the chamber may be directly exposed to an outer surface of the first spike component. Alternatively, the first spike component can include a channel. The channel can fluidly connect the chamber. The channel can fluidly connect to the outer surface of the first spike component. Thus, the channel can fluidly connect the chamber to the outer surface of the first spike component.

The coupling component, e.g. sleeve, can cover the channel to prevent release of the active drug substance. In one or more exemplary drug delivery devices, the sleeve can further include a mating protrusion, e.g. mating feature, mating extension. The mating protrusion may be configured to at least partially insert into the channel and block the channel. Thus, the mating protrusion may form a fluid tight seal to prevent release of the active drug substance. The mating protrusion may be flexible in order to provide a seal. The mating protrusion may be rigid. The channel may include a mating feature configured to mate with the mating protrusion, thus preventing the active drug substance from releasing.

The mating protrusion can advantageously be used while allowing the sleeve to be looser, e.g. the sleeve may not form a fluid-tight connection with the first spike component. The mating protrusion can prevent any rotational or longitudinal motion of the sleeve while inserted into the channel. The mating protrusion can act as a plug or cork to close the channel. The mating protrusion can advantageously allow for the sleeve to dissolve before the mating protrusion, which can delay release of the active drug substance. For example, as discussed below, the sleeve may dissolve first to release the first spike component from the carrier. After a time, the mating protrusion can dissolve to release the active drug substance. However, the sleeve and mating protrusion may also dissolve at the same time.

The above-description mentions that certain components may be fluid tight, e.g. have a fluid tight connection. Alternatively, fluid tight may mean closed, gas tight, active drug substance tight, and/or sealed.

The fluid-tight connection can include 100, 99, 98, 97, 96, 95, 90, or 85% fluid tight. The fluid-tight connection can include less than 100, 99, 98, 97, 96, 95, 90, or 85% fluid tight. This may include greater than 99, 98, 97, 96, 95, 90, or 85% fluid tight. The fluid-tight connection can mean that a seal is formed to prevent release of an active drug substance.

In one or more exemplary drug delivery devices, the fluid-tight connection may only last for a period of time. For example, any of the components can undergo physical changes, such as shape changes and/or dissolving, that could break the fluid tight connection after a given time period.

Advantageously, the coupling component/sleeve can be used to prevent release of the active drug substance from the chamber in the first spike component. A number of different methods can be used to release the active drug substance.

In one or more exemplary drug delivery devices, the coupling component/sleeve can be dissolvable. After a period of time the coupling component/sleeve can dissolve, thus providing an opening in the first spike component for the active drug substance to be released. In one or more exemplary delivery devices, a portion of the coupling component/sleeve can dissolve to release the active drug substance while the first spike component remains held within the remainder of the coupling component/sleeve. In alternative drug delivery devices, the coupling component/sleeve can fully dissolve to release the first spike component.

In one or more exemplary drug delivery devices, the first spike component can be movable, e.g. translatable within the coupling component/sleeve. Thus, the first spike component is configured to translate with respect to the coupling component/sleeve. For example, the first spike component can move longitudinally within the coupling component/sleeve. For example, the first spike component can move within the coupling component/sleeve during rotation of the first body part. The rotational force may move the first spike component. The first spike component can move in the coupling component/sleeve during the stopping of the connector component after rotation.

In one or more exemplary drug delivery systems, the first spike component can move distally, e.g. towards the first distal end of the first spike component. Thus, the coupling component/sleeve will move relatively proximally as compared to its original position. This can expose the chamber, thereby releasing the active drug substance.

In one or more exemplary drug delivery systems, the first spike component can move proximally, e.g. towards the proximal end of the first spike component. Thus, the coupling component/sleeve will move relatively distally as compared to its original position. This can expose the chamber, thereby releasing the active drug substance.

In one or more exemplary drug delivery systems, the first spike component can translate completely out of the coupling component/sleeve, thus releasing from drug delivery device. Alternatively, the first spike component can remain attached to the coupling component/sleeve after the movement. For example, the first spike component may have a proximal end wider than the coupling component/sleeve, and thus the first spike component can be prevented from releasing from the coupling component/sleeve.

In one or more exemplary drug delivery devices, the coupling component/sleeve can be dissolvable or partially dissolvable and the first spike component can be configured to translate with respect to the coupling component/sleeve. In one or more exemplary drug delivery devices, the coupling component/sleeve can be dissolvable or partially dissolvable or the first spike component can be configured to translate with respect to the coupling component/sleeve. In one or more exemplary drug delivery devices, the coupling component/sleeve can be dissolvable or partially dissolvable and/or the first spike component can be configured to translate with respect to the coupling component/sleeve.

While the above-description can be advantageously used for releasing the active drug substance at a particular time, it can also be useful for releasing the first spike component from the remainder of the drug delivery device, e.g. the carrier. This way, the first spike component can remain in the patient while the remainder of the drug delivery device, e.g., carrier, can be released and exit the patient. The drug delivery device can then be retrieved while the active drug substance can still be delivered.

In one or more exemplary drug delivery devices, as mentioned above, the coupling component/sleeve may fully dissolve. This can release the first spike component from the connector component. The first spike component can then remain in the patient while the remainder of the drug delivery device, e.g. carrier, is released.

In one or more exemplary drug delivery devices, as mentioned above, the first spike component can translate fully out of the coupling component/sleeve. This can release the first spike component from the connector component. The first spike component can then remain in the patient while the remainder of the drug delivery device, e.g. carrier, is release.

In one or more exemplary drug delivery devices, the first spike may be a single unit, e.g. unitary. Alternatively, the first spike may be formed from multiple components. For example, the first spike may be formed from a proximal spike component and a distal spike component. The proximal spike component may have a proximal spike mating feature on its distal end. The distal spike component may have a distal spike mating feature on its proximal end. Thus, the first spike component comprises a distal spike component having a distal spike mating feature and a proximal spike component having a proximal spike mating feature. The proximal spike mating feature can mate with the distal spike mating feature to form the full first spike component. The mating can be performed mechanically and/or chemically. More components can form the first spike component, such as 2, 3, 4, 5, or 6 components.

In one or more exemplary drug delivery devices, the first spike component may be breakable into a proximal spike component and a distal spike component. For example, the first spike component may be scored for ease of breakage into a proximal spike component and a distal spike component. The first spike component can include a material that is softer and/or dissolvable, for releasing a proximal spike component from a distal spike component. This material may act as a separator between the proximal spike component and the distal spike component.

Advantageously, the distal spike component may contain the chamber, and the active drug substance. Thus, the distal spike component can be released, e.g. separated, from the proximal spike component and the distal spike component can remain in the patient for delivering the active drug substance while the remainder of the drug delivery device passes through the patient.

In one or more exemplary drug delivery devices, the coupling component/sleeve, may be used to keep the proximal spike component connected to the distal spike component. The coupling component/sleeve can also be used for other additional reasons as discussed above. For example, the coupling component/sleeve can also close the chamber as previously discussed.

In one or more exemplary drug delivery devices, the coupling component/sleeve can be dissolvable. After a period of time the coupling component/sleeve can dissolve, thus providing both an opening for the active drug substance to be released as well as allow for uncoupling, e.g. separation, of the proximal spike mating feature from the distal spike mating feature, releasing the distal spike component from the proximal spike component, and thus the carrier.

In one or more exemplary delivery devices, a portion of the coupling component/sleeve can dissolve to release the distal spike component with active drug substance while the proximal spike component remains held within the coupling component/sleeve. In alternative drug delivery devices, the coupling component/sleeve can fully dissolve to release the first spike component, which can separate into the proximal spike component and the distal spike component. In an alternate drug delivery device, a portion of the coupling component/sleeve can dissolve, releasing the distal spike component, while a remainder of the coupling component/sleeve can still cover the chamber. This remainder can then later dissolve to release the active drug substance.

In one or more exemplary drug delivery devices, the first spike component can be movable, e.g. translatable within the coupling component/sleeve. For example, the first spike component can move longitudinally within the coupling component/sleeve component. The first spike component can move during rotation of the first body part. The first spike component can move during stopping of the connector component after rotation.

In one or more exemplary drug delivery systems, the first spike component can move distally, e.g. towards the first distal end of the first spike component. Thus, the coupling component/sleeve will move relatively proximally as compared to its original position. This can expose the chamber, thereby releasing the active drug substance. This can also expose the proximal spike mating feature and the distal spike mating feature, allowing for release of the distal spike component.

In one or more exemplary drug delivery systems, the first spike component can move proximally, e.g. towards the first proximal end of the first spike component. Thus, the coupling component/sleeve will move relatively distally as compared to its original position. This can expose the chamber, thereby releasing the active drug substance. This can also expose the proximal spike mating feature and the distal spike mating feature, allowing for release of the distal spike component.

In one or more exemplary drug delivery systems, the first spike component can translate completely out of the coupling component/sleeve, thus releasing the first spike component to be separated into the proximal spike component and the distal spike component.

Alternatively, the proximal spike component can remain attached to the coupling component/sleeve after the movement, while the distal spike component is released. For example, the proximal spike component may have a proximal end wider than the coupling component/sleeve, and thus the proximal spike component can be prevented from releasing from the coupling component/sleeve.

The present disclosure also discloses a drug delivery device having a central axis, the drug delivery device comprising a first body part; a second body part; an actuator mechanism configured to rotate at least one of the first body part or the second body part with respect to one another about the central axis. The drug delivery device comprises a first attachment part comprising a connector component and/or a first spike component. The connector component may be connected to the first body part and the first spike component. The connector component is optionally configured to separate from the first body part.

The connector component can be optionally configured to separate, such as translate away, break apart, from the first body part. For example, the entire connector component can separate from the first body part. In one or more example drug delivery devices, the connector component can fully separate from the first body part, and no portion of the connector component can remain attached to the first body part.

Alternatively, a portion, such as a section, piece, of the connector component can separate from, such as translate away, break apart, the first body part, leaving another portion of the connector component attached to the first body part.

Both variations, and combinations in between, indicate that the connector component is configured to separate from the first body part.

As discussed above, in one or more exemplary drug delivery systems, the first spike component can be released from one or more of the coupling component, sleeve component, and connector component. Alternatively, or in conjunction with the above, a portion or all of the connector component can be released from the first body part. The first spike component may remain connected to the connector component, such as directly or via an intermediate component such as the coupling component, or can be released as discussed above.

In one or more exemplary drug delivery systems, the connector component is configured to separate from the first body part. As discussed herein, separating can include the connector component being configured to break apart from, break away from, release from, remove from, or translate away from, the first body part. Advantageously, a reminder of the drug delivery system can continue to pass through a patient's system while the connector component remains for delivery of an active drug substance.

In one or more exemplary drug delivery systems, the connector component can be configured to break into a first connector section and a second connector section. The second connector section can be configured to remain attached to the first body part. The particular portion of the connector component making up the first connector section and/or the second connector section is not limiting. Alternatively, the connector component can fully separate from the first body part, and may not break into a first connector section and a second connector section.

In one or more exemplary drug delivery systems, the connector component can include a pre-weakened area (e.g., pre-weakened line, pre-weakened section, pre-weakened plane, pre-weekend component). The pre-weakened area can be configured to break (or separate) upon an application of a sufficient force.

In one or more exemplary drug delivery systems, the pre-weakened area can be physically weakened. The pre-weakened area can be a thinned area (e.g., the material may be thinner at the pre-weakened area). The pre-weakened area can be a scored area. The particular type of weakening is not limiting. The pre-weakened area can be configured to break upon application of a sufficient force.

In one or more exemplary drug delivery systems, the pre-weakened area can be a biodegradable area. Thus, after a period of time the pre-weakened area may be degraded for release of the connector component from the first body part. For example, the pre-weakened area can be water soluble. The pre-weakened area can include a water soluble area. The pre-weakened area can be fat soluble.

In one or more exemplary drug delivery systems, the pre-weakened area can be a combination of a physical weakening and a biodegradable portion.

The pre-weakened area can be located between the first connector section and the second connector section. This can allow for separation of the first connector section from the second connector section.

In one or more exemplary drug delivery systems, the pre-weakened area can be located at an attachment point between the connector component and the first body part. This can allow for separation of the connector component from the first body part. Thus, the connector component may not separate into a first connector section and a second connector section, but may instead completely separate from the first body part.

In one or more exemplary drug delivery systems, the pre-weakened area can be configured to break (allowing for separation of the connector component from the first body part) during or immediately after rotation of the first body part with respect to the second body part. As there are increased forces on the connector component during this processes, this can be sufficient to break the pre-weakened area.

In one or more exemplary drug delivery systems, the pre-weakened area can be configured to break (allowing for separation of the connector component from the first body part) during rotation of the first body part with respect to the second body part. Thus, the rotation may provide sufficient force to separate the connector component from the first body part.

Accordingly, the connector component can be configured to separate from the first body part during rotation of the first body part and the second body part.

In one or more exemplary drug delivery systems, the pre-weakened area can be configured to break (allowing for separation of the connector component from the first body part) upon stoppage of rotation of the first body part. Thus, the stopping may provide sufficient force to separate the connector component from the first body part. Accordingly, the connector component can be configured to separate from the first body part upon stoppage of rotation of the first body part and the second body part. The stopping may occur, for example, upon penetration of tissue. Advantageously, this may prevent overpenetration of tissue.

In one or more exemplary drug delivery systems, the connector component can include a high friction surface (e.g., a surface that has a higher friction than the adjacent portions of the connector component). The high friction surface can be, for example, one or more of: bumps, roughening, protrusions, grooves, and corrugation. The high friction surface can cover a portion of the connector component. The high friction surface can cover all of the connector component. The high friction surface can be located on one side of the connector component. The high friction surface can be located on the first spike component. The high friction surface may, for example, resist movement of the connector component with respect to the remainder of the drug delivery device, increasing forces on the connector component.

In one or more exemplary drug delivery devices, the carrier comprises a second body part. The second body part may be a two-part body part, i.e. the second body part may comprise a second primary body part and a second secondary body part. The second body part may include and/or all of the features discussed above with respect to the first body part. The second body part may be a mirror image of the first body part.

The second body part may include one or more of the components discussed herein, such as a second attachment part. The second attachment part may be fixed or rotationally attached to the second body part. In one or more exemplary drug delivery devices, the second body part has an outer surface.

In one or more exemplary drug delivery devices, the carrier can include an actuator mechanism. The actuator mechanism is configured to rotate the first body part in relation to the second body part about a primary axis of the drug delivery device. The primary axis may be parallel to and/or coinciding with the central axis.

In one or more exemplary drug delivery devices, the first body part is configured to rotate in a first direction and/or the second body part is configured to rotate in a second direction opposite to the first direction.

The drug delivery device may comprise a frame part, where different parts, such as the first body part and/or the second body part are attached, e.g., fixed or rotatably attached to the frame part. In one or more exemplary drug delivery devices, the actuator mechanism, or parts thereof, may be attached to the frame part. Thereby, separate rotation of the first body part and the second body part in relation to the frame part may be provided for.

The rotational connection between the first body part and the second body part allows the first body part to rotate relative to the second body part, without the two parts separating from each other before the attachment part(s) interact with the internal tissue, such as mucous membranes. Such a connection may be obtained in a plurality of ways, where in one example the first body part has a plug connection and the second body part has a socket connection, where this plug and socket configuration allows the first body part to rotate relative to the second body part. A second example could be to provide an axle that may be coaxial with the central axis and/or the primary axis, where the first body part and the second body part are configured to receive the axle, and a stopping device is arranged at first and second ends of the axle, on each side of the combined first and second body part, preventing the first body part and the second body part to slide in a longitudinal direction along the axle. The axle may be integrated in the first body part or in the second body part.

The first and/or the second body part may be arranged to rotate freely relative to each other, e.g., at least in the second state, and thereby allowing the attachment parts to rotate relative to each other. Thus, the attachment parts may be adapted to come into contact and/or penetrate tissue of the gastrointestinal tract. The rotation of the body parts relative to each other using a resilient force may move the attachment parts in such a way that they are capable of e.g., penetrating or pinching the mucous membrane in order to fix the drug delivery device at a location in the gastrointestinal tract, such as the stomach or intestines. The penetrating and/or pinching force may come from the actuator mechanism/resilient part, where the resilient part may be adapted to store a resilient force that is capable of forcing the attachment parts towards each other when the resilient force of the resilient part has been at least partly unleashed. The resilient part may e.g., be in the form of a spring or spring element, for example a torsional spring or a power spring, where the spring may be wound up to store mechanical energy, where the mechanical energy may be transmitted to the first and/or the second body part. When the mechanical energy is released, the first body part may rotate relative to the second body part, and where the mechanical energy may be transferred into the attachment parts via the body parts.

Within the context of the present description the term “rotational force” may be seen as torque, moment, moment of force, rotational force or “turning effect”. Another definition of the term “rotational force” may be the product of the magnitude of the force and the perpendicular distance of the line of action of force from the axis of rotation. The rotational force may be seen as the force which is transferred from the resilient part to the attachment members of the drug delivery device via the body parts.

The rotational force may be defined as being large enough to penetrate into the gastrointestinal tissue. When the rotational force is applied to both the first and the second body part, the first attachment member may come into contact with the surface to be attached to, and where the rotational force applied to the second body part may cause the second attachment part to come into contact with the same surface, where the first attachment part provides a force, while the second attachment part provides a counter force to the first attachment part, so that the force is applied in such a manner that the first attachment part is forced in a direction towards the second attachment part, or vice versa.

In one or more exemplary drug delivery devices, a distance between the first attachment axis of the first attachment part and the primary axis, e.g., at least in an activated state or second state of the drug delivery device and optionally in an initial state of the drug delivery device, is larger than 0.5 mm.

In one or more exemplary drug delivery devices, a distance between the second attachment axis of the second attachment part and the primary axis, e.g., at least in an activated state or second state of the drug delivery device and optionally in an initial state of the drug delivery device, is larger than 0.5 mm.

In one or more exemplary drug delivery devices, the first attachment part is rotationally attached to the first body part, e.g. via a first joint connection having a first rotation axis. In other words, the first attachment part is optionally configured to rotate about a first rotation axis, e.g. in relation to the first body part. The first rotation axis may be parallel to the central axis and/or the primary axis. The first rotation axis may form a first angle with the central axis and/or the primary axis. The first angle may be less than 15°. The first angle may be in the range from 75° to 105°, such as 90°+5° or 90°.

In one or more exemplary drug delivery devices, the first body part may define a first recess (e.g., cavity, gap, slot, hole, aperture, retainer, first body recess) extending to an outer surface of the first body part. The first recess may be formed by solid walls on all sides except an outermost surface, which is open. For example, the first recess may be formed by a first wall, a second wall, and a bottom. The first wall may be opposite the second wall. The bottom may connect the first wall and the second wall. In one or more exemplary drug delivery devices, the first wall and/or the bottom wall and/or the second wall are flat. In one or more exemplary drug delivery devices, the first wall and/or the bottom wall and/or the second wall are curved. The open outermost surface may be curved to follow along with an outer surface of the carrier.

As mentioned above, the first attachment part may be rotationally connected within the first recess. For example, the first attachment part may be rotationally connected within the first recess along a first attachment part axis. The first attachment part axis may be, for example, a pin (e.g., arm, support). The first attachment part axis may be aligned with the central axis and/or the primary axis. The first attachment part axis may be parallel to the central axis and/or the primary axis. The first attachment part axis may be angled with respect to the central axis and/or the primary axis. Accordingly, the first attachment part can rotate within the recess along the first attachment part axis. Further, rotation of the first attachment part may be stopped at end surfaces of the recess.

The first recess may extend along a portion of the outer surface of the first body. The first recess may extend fully along an outer circumference of the first body. The first recess may extend around 10, 15, 20, 25, 30, 35, 40, 45, 50, 55, 60, 65, 70, 75, 80, 85, 90, 95, or 100% of an outer circumference of the first body. The first recess may extend around greater than 10, 15, 20, 25, 30, 35, 40, 45, 50, 55, 60, 65, 70, 75, 80, 85, 90, or 95% of an outer circumference of the first body. The first recess may extend around less than 10, 15, 20, 25, 30, 35, 40, 45, 50, 55, 60, 65, 70, 75, 80, 85, 90, 95, or 100% of an outer circumference of the first body. The first body may optionally contain more than one first recess, for example of a plurality of first attachment parts are used on the first body. If more than one first recess is used, they may be spaced longitudinally apart and/or circumferentially apart.

The first recess may extend from an outer surface toward the central axis through 5, 10, 15, 20, 25, 30, 35, or 40% of the drug delivery device. The first recess may extend from an outer surface toward the central axis through greater than 5, 10, 15, 20, 25, 30, 35, or 40% of the drug delivery device. The first recess may extend from an outer surface toward the central axis through less than 5, 10, 15, 20, 25, 30, 35, or 40% of the drug delivery device.

In one or more exemplary drug delivery devices, the first recess may extend circumferentially, or partially circumferentially around the first body with the central axis being the longitudinal direction. The first recess may extend perpendicularly with respect to the central axis and/or the primary access (e.g., may extend along a cross section of the drug delivery device perpendicular to the central axis and/or the primary access).

The first recess may have any number of shapes when viewed in cross section with respect to the first attachment part axis. For example, the first recess can be a portion of a circle, such as a half circle. The first recess can be a triangle. The first recess can be a sector of a circle. The first recess can be generally circular in shape, with a portion of the circle cut off. For example, the portion of the circle can be cut off from two points on the circumference of the circle connected by a single straight line. The first recess can be a curved edge connected by two straight edge. The first recess can be two curved edges connected to each other by two straight edges.

For example, in one or more exemplary drug delivery systems, the first recess can form a slice of the first body part. Thus, the first recess can extend greater circumferentially than longitudinally.

In alternate drug delivery devices, the first recess is a cylinder. In alternate drug delivery devices, the first recess is a rectangular prism.

The first recess may be relatively thin. For example, the first recess may be the longitudinal length of the first spike component. The first recess may have a longitudinal length slightly longer than the first spike component.

The first recess may be parallel to the central axis and/or the primary access. The first recess may be orthogonal to the central axis and/or the primary access. In one or more exemplary drug delivery devices, the first recess extends perpendicular to the central axis. The first recess may be at an angle of 5, 10, 15, 20, 25, 30, 35, 40, 45, 50, 55, 60, 65, 70, 75, 80, or 85 degrees with respect to the central axis and/or the primary access. The first recess may be at an angle of greater than 5, 10, 15, 20, 25, 30, 35, 40, 45, 50, 55, 60, 65, 70, 75, 80, or 85 degrees with respect to the central axis and/or the primary access. The first recess may be at an angle of less than 5, 10, 15, 20, 25, 30, 35, 40, 45, 50, 55, 60, 65, 70, 75, 80, or 85 degrees with respect to the central axis and/or the primary access.

The first body attachment part axis may be parallel to the central axis and/or the primary access. The first body attachment part axis may be orthogonal to the central axis and/or the primary access. The first body attachment part axis may be at an angle of 5, 10, 15, 20, 25, 30, 35, 40, 45, 50, 55, 60, 65, 70, 75, 80, or 85 degrees with respect to the central axis and/or the primary access. The first body attachment part axis may be at an angle of greater than 5, 10, 15, 20, 25, 30, 35, 40, 45, 50, 55, 60, 65, 70, 75, 80, or 85 degrees with respect to the central axis and/or the primary access. The first body attachment part axis may be at an angle of less than 5, 10, 15, 20, 25, 30, 35, 40, 45, 50, 55, 60, 65, 70, 75, 80, or 85 degrees with respect to the central axis and/or the primary access.

Thus, the first attachment part may rotate on the first attachment part axis in order to move perpendicular to the central axis and/or the primary axis. In certain embodiments, the first attachment part may rotate at an angle between perpendicular and parallel with respect to the central axis and/or the primary axis.

In one or more exemplary drug delivery devices, when the first body part and/or the second body part rotate with respect to one another, the first attachment part and/or the second attachment part can rotate out of their respective recesses (e.g., first recess and second recess) due to the rotation of the first body part and/or the second body part. The continued rotation of the first body part and/or the second body part then causes the first attachment part and/or the second attachment part to pierce tissue to hold the drug delivery device in place. Thus, in a first state, the first attachment part may be located within the first recess. After rotation to a second state, the first attachment part may be located at least partially outside of the first recess.

In one or more exemplary drug delivery devices, the first attachment part extends, e.g. at least in an activated state of the drug delivery device and optionally in an initial state of the drug delivery device, in a direction away from the first body part. In other words, the first spike component may extend, e.g. at least in an activated state of the drug delivery device and optionally in an initial state, from an outer surface of the first body part. Formulated differently, the first attachment axis may, e.g. at least in an activated state of the drug delivery device and optionally in an initial state of the drug delivery device form an angle of at least 45° with the central axis and/or the primary axis. An attachment part extending in a direction is to be understood as the direction from proximal end of attachment part/spike component part to distal end of attachment part along the attachment axis of the attachment part.

The first attachment part may in a first state of the drug delivery device extend in a first primary direction and in a second state of the drug delivery device extend in a first secondary direction. The first primary direction and the first secondary direction may form an angle of at least 30°. The first primary direction may be parallel or substantially parallel to the central axis. The first primary direction may form an angle less than 60° with the central axis.

The first secondary direction may form an angle of at least 60° such as about 90° with the central axis. The first secondary direction may be perpendicular to the central axis.

The first distal end of the first attachment part may be configured to move or be moved from a first primary position in a first state of the drug delivery device to a first secondary position in the second state.

The drug delivery device comprises a second attachment part located on the second body part. The second attachment part may comprise a second base part and/or a second spike component. The second attachment part has a second proximal end and a second distal end. The second attachment part, such as the second spike component, optionally has or extends along a second attachment axis. A second tip of the second spike component forms the second distal end. In other words, the second distal end is a second tip of the second spike component. The second base may be arranged at or constitute the second proximal end of the second attachment part. The second spike component may have a length in the range from 1 mm to 15 mm such, as in the range from 3 mm to 10 mm. Thereby sufficient penetration into the internal tissue may be provided for while at the same time reducing the risk of damaging the internal tissue. The second distal end of the second attachment part may be provided with a tip configured to penetrate a biological tissue. The second distal end of the second attachment part may be provided with a gripping part configured to grip a biological tissue.

The second spike component may be similar to the first spike component, and can include any or all of the features discussed above with respect to the first spike component. For example, the second spike component may include a chamber for holding an active drug substance. The second spike component may be formed of multiple components, e.g. proximal second spike component and a distal second spike component. Alternatively, the second spike component may be unitary and not include a chamber.

The second attachment part may be similar to the first attachment part, and can include any or all of the features discussed above with respect to the first spike component. For example, the second attachment part may include a second coupling component including any or all of the features of the coupling component discussed above. Alternatively, the second attachment part may not include the coupling component.

The second spike component may have a cross-sectional diameter in the range from 0.1 mm to 5 mm, such as in the range from 0.5 mm to 2.0 mm.

The second spike component may be straight and/or curved. The second spike component may comprise a second primary section that is straight. The second spike component may comprise a second secondary section, e.g. between the second primary section and the second distal end or between the second base and the second primary section. The second secondary section may be curved.

Alternatively, the second spike component may include two or more straight portions formed at an angle. For example, the second spike component may have a proximal portion that extends at a first angle from a connection point to the drug delivery device and a distal portion that extends at a second angle from a connection point to the drug delivery device. The first angle and the second angle may be different. The proximal portion may connect to the distal portion at a joint (e.g., bend, connection, angle) and have a joint angle between the proximal portion and the distal portion. The joint angle may be an acute angle, an obtuse angle, or a right angle. The angle may be, for example, 10, 20, 30, 40, 50, 60, 70, 80, 90, 100, 120, 130 140, 150, 160, or 170 degrees. This can advantageously allow for different angles of attach when the first spike component interacts with inner surface linings. This may allow for improved attachment of the drug delivery device, while helping to reduce or avoid tissue damage. Further, the joint may be flexible. Alternatively, the joint may not be flexible.

The joint may be located at a center, or generally at a center, of a length of the second spike component. Alternatively, the joint may be located 40, 45, 55, 60, or 65% up a length of the second spike component from the proximal end.

In alternative embodiments, the second spike component may have three, four, or five different portions at different angles, each connected by a joint. In some iterations, any or all of the different portions may be straight or curved. Each joint may be flexible or not flexible.

In one or more exemplary drug delivery devices, both the first second and the second spike component include a joint. However, only one of the first spike component and the second spike component may include a joint with the other being straight and/or curved. If both the first spike component and the second spike component include a joint, the first distal tip and the second distal tip may be angled towards one another in order to facilitate attachment when the first body part and the second body part rotate with respect to one another.

In one or more exemplary drug delivery devices, the second attachment part is rotationally attached to the second body part, e.g. via a second joint connection having a second rotation axis. In other words, the second attachment part is optionally configured to rotate about a second rotation axis (or second attachment part axis), e.g. in relation to the second body part. The second rotation axis may be parallel to the central axis and/or the primary axis. The second rotation axis may form a second angle with the central axis and/or the primary axis. The second angle may be less than 15°. The second angle may be in the range from 75° to 105°, such as 90°+5° or 90°.

In one or more exemplary drug delivery devices, the second body part may define a second recess (e.g., cavity, gap, slot, hole, aperture, retainer) extending to an outer surface of the second body part. The second recess may be formed by solid walls on all sides except an outermost surface which is open. For example, the second recess may be formed by a first wall, a second wall, and a bottom. The first wall may be opposite the second wall. The bottom may connect the first wall and the second wall. In one or more exemplary drug delivery devices, the first wall and/or the bottom wall and/or the second wall are flat. In one or more exemplary drug delivery devices, the first wall and/or the bottom wall and/or the second wall are curved. The open outermost surface may be curved to follow along with an outer surface of the carrier.

The second attachment part may be rotationally connected within the second recess. For example, the second attachment part may be rotationally connected within the second recess along a second attachment part axis. The second attachment part axis may be, for example, a pin (e.g., arm, support). The second attachment part axis may be aligned with the central axis and/or the primary axis. The second attachment part axis may be parallel to the central axis and/or the primary axis. The second attachment part axis may be angled with respect to the central axis and/or the primary axis. Accordingly, the second attachment part can rotate within the second recess along the second attachment part axis. Further, rotation of the second attachment part may be stopped at end surfaces of the second recess.

The second recess may extend along a portion of the outer surface of the second body part. The second recess may extend fully along an outer circumference of the second body part. The second recess may extend around 10, 15, 20, 25, 30, 35, 40, 45, 50, 55, 60, 65, 70, 75, 80, 85, 90, 95, or 100% of an outer circumference of the second body part. The second recess may extend around greater than 10, 15, 20, 25, 30, 35, 40, 45, 50, 55, 60, 65, 70, 75, 80, 85, 90, or 95% of an outer circumference of the second body part. The second recess may extend around less than 10, 15, 20, 25, 30, 35, 40, 45, 50, 55, 60, 65, 70, 75, 80, 85, 90, 95, or 100% of an outer circumference of the second body part. The second body part may optionally contain more than one second recess, for example of a plurality of second attachment parts are used on the second body part.

The second recess may extend from an outer surface toward the central axis through 5, 10, 15, 20, 25, 30, 35, or 40% of the drug delivery device. The second recess may extend from an outer surface toward the central axis through greater than 5, 10, 15, 20, 25, 30, 35, or 40% of the drug delivery device. The second recess may extend from an outer surface toward the central axis through less than 5, 10, 15, 20, 25, 30, 35, or 40% of the drug delivery device.

In one or more exemplary drug delivery devices, the second recess may extend circumferentially, or partially circumferentially around the second body part with the central axis being the longitudinal direction. The second recess may extend perpendicularly with respect to the central axis and/or the primary access (e.g., may extend along a cross section of the drug delivery device perpendicular to the central axis and/or the primary access).

The second recess may have any number of shapes when viewed in cross section with respect to the second attachment part axis. For example, the second recess can be a portion of a circle, such as a half circle. The second recess can be a triangle. The second recess can be a sector of a circle. The second recess can be generally circular in shape, with a portion of the circle cut off. For example, the portion of the circle can be cut off from two points on the circumference of the circle connected by a single straight line. The second recess can be a curved edge connected by two straight edge. The second recess can be two curved edges connected to each other by two straight edges.

For example, in one or more exemplary drug delivery systems, the second recess can form a slice of the second body part. Thus, the second recess can extend greater circumferentially than longitudinally.

In alternate drug delivery devices, the second recess is a cylinder. In alternate drug delivery devices, the second recess is a rectangular prism.

The second recess may be relatively thin. For example, the second recess may be the longitudinal length of the second spike component. The second recess may have a longitudinal length slightly longer than the second spike component.

The second recess may be parallel to the central axis and/or the primary access. The second recess may be orthogonal to the central axis and/or the primary access. The second recess may be at an angle of 5, 10, 15, 20, 25, 30, 35, 40, 45, 50, 55, 60, 65, 70, 75, 80, or 85 degrees with respect to the central axis and/or the primary access. The second recess may be at an angle of greater than 5, 10, 15, 20, 25, 30, 35, 40, 45, 50, 55, 60, 65, 70, 75, 80, or 85 degrees with respect to the central axis and/or the primary access. The second recess may be at an angle of less than 5, 10, 15, 20, 25, 30, 35, 40, 45, 50, 55, 60, 65, 70, 75, 80, or 85 degrees with respect to the central axis and/or the primary access.

The second body attachment part axis may be parallel to the central axis and/or the primary access. The second body attachment part axis may be orthogonal to the central axis and/or the primary access. The second body attachment part axis may be at an angle of 5, 10, 15, 20, 25, 30, 35, 40, 45, 50, 55, 60, 65, 70, 75, 80, or 85 degrees with respect to the central axis and/or the primary access. The second body attachment part axis may be at an angle of greater than 5, 10, 15, 20, 25, 30, 35, 40, 45, 50, 55, 60, 65, 70, 75, 80, or 85 degrees with respect to the central axis and/or the primary access. The second body attachment part axis may be at an angle of less than 5, 10, 15, 20, 25, 30, 35, 40, 45, 50, 55, 60, 65, 70, 75, 80, or 85 degrees with respect to the central axis and/or the primary access.

Thus, the second attachment part may rotate on the second attachment part axis in order to move perpendicular to the central axis and/or the primary axis. In certain embodiments, the second attachment part may rotate at an angle between perpendicular and parallel with respect to the central axis and/or the primary axis.

In one or more exemplary drug delivery devices, when the first body part and/or the second body part rotate with respect to one another, the first attachment part and/or the second attachment part can rotate out of their respective recesses (e.g., first recess and second recess) due to the rotation of the first body part and/or the second body part. The continued rotation of the first body part and/or the second body part then causes the first attachment part and/or the second attachment part to pierce tissue to hold the drug delivery device in place. Thus, in a first state, the second attachment part may be located within the second recess. After rotation to a second state, the second attachment part may be located at least partially outside of the second recess.

In one or more exemplary drug delivery devices, the second attachment part includes a second connector component, e.g. arm, holder, extension. In one or more exemplary drug delivery devices, second first attachment part includes a second spike component. The second spike component can be attached at a distal end of the second connector component. The second spike component can be mechanically attached to the distal end of the second connector component. The second spike component can be chemically attached to the distal end of the second connector component. The second spike component can be integral with the second connector component. The second spike component can be permanently attached to the distal end of the second connector component. The second spike component can rotate with respect to the second connector component, such as on an axis at the distal end of the second connector component. The second spike component may not rotate with respect to the second connector component. The second spike component can be removably attached to the distal end of the second connector component. A proximal end of the second connector component can be associated with the second attachment part axis. Thus, the second connector component and second spike component can rotate with respect to the second attachment part axis.

The second connector component may be straight. The second connector component may be bent. The second connector component may include a number of bends. The second connector component may be curved. The second connector component can be a single unit. The second connector component can be a number of segments attached together. The number of segments may be rigidly attached together. The number of segments may be rotatably attached.

The second connector component can optionally include further intermediate components.

In one or more exemplary drug delivery devices, the second attachment part includes a second connector component (e.g., arm). In one or more exemplary drug delivery devices includes a second spike component (e.g., spike). The second spike component can be attached at a distal end of the second connector component. The second spike component can be mechanically attached to the distal end of the second connector component. The second spike component can be chemically attached to the distal end of the second connector component. The second spike component can be integral with the second connector component. The spike can be permanently attached to the distal end of the second connector component. The second spike component can be removably attached to the distal end of the second connector component.

In one or more exemplary drug delivery devices, the second connector component can include a spike holder. The spike holder can be located on the second connector component distal end. The second spike component can be attached to the spike holder.

In one or more exemplary drug delivery devices, the spike holder can be a cylinder. The spike holder may have an aperture (e.g., hole, lumen, bore, through hole) extend through a length, such as a longitudinal length) of the spike holder. The aperture may be configured to receive and retain the second spike component. Thus, the second spike component can be retained within the spike holder, which can be attached to the second connector component. For example, a distal end of the second connector component could include a cradle (e.g., seat, holder, slot, receiver, mating element) configured to conform with the spike holder. Alternatively, a distal end of the second connector component could include a cradle (e.g., seat, holder, slot, receiver, mating element) configured to conform with the second spike component.

As mentioned, the second spike component may contain an active drug substance. For example, the second spike component may contain a chamber for holding the active drug substance. The chamber may be, for example, a through-going bore. The second spike component may not contain an active drug substance.

In one or more exemplary drug delivery devices, the second spike component may be releasable from the spike holder. For example, the spike holder may be dissolvable. The spike holder can be configured to be dissolvable at a specific time period. Once the spike holder is partially or fully dissolved, the second spike component can be released from a remainder of the drug delivery device. Upon release of the second spike component, the remainder of the delivery system can release from the patient while the second spike component remains embedded within the patient. Thus, the drug delivery device can be retrieved and the process of reusing it can begin while the second spike component continues to provide an active drug substance to the patient. This can further reduce or eliminate any accidental release of the delivery system with the active drug substance from the patient.

A proximal end of the second connector component can be associated with the second attachment part axis. Thus, the second connector component and second spike component can rotate with respect to the second attachment part axis. Thus, the second connector component is rotatable within the second recess about a second rotation axis (e.g., the second attachment part axis), wherein the second recess is formed by two opposing sidewalls of the second body part.

In one or more exemplary drug delivery devices, the second body part may not contain a second recess. For example, the second attachment part, be it the second spike component along or the second connector component and spike combination, may be directly attached to an outer surface of the second body part.

In one or more exemplary drug delivery devices, the second attachment part extends, e.g. at least in an activated state of the drug delivery device and optionally in an initial state of the drug delivery device, in a direction optionally away from the second body part. In other words, the second spike component may extend, e.g. at least in an activated state of the drug delivery device and optionally in an initial state, from an outer surface of the second body part. Formulated differently, the second attachment axis may, e.g. at least in an activated state of the drug delivery device and optionally in an initial state of the drug delivery device form an angle of at least 45° with the central axis and/or the primary axis.

The second attachment part may in a first state of the drug delivery device extend in a second primary direction and in a second state of the drug delivery device extend in a second secondary direction. The second primary direction and the second secondary direction may form an angle of at least 30°. The second primary direction may be parallel or substantially parallel to the central axis. The second primary direction may form an angle less than 60° with the central axis. The second secondary direction may form an angle of at least 60°, such as about 90° with the central axis. The second secondary direction may be perpendicular to the central axis.

The second distal end of the second attachment part may be configured to move or be moved from a second primary position in a first state of the drug delivery device to a second secondary position in the second state.

In one or more exemplary drug delivery devices, the drug delivery device comprises a third attachment part. The third attachment part can be located on the first body part and/or the second body part. The third attachment part may comprise a third base part and/or a third spike component. The third attachment part has a third proximal end and a third distal end. The third attachment part, such as the third spike component, optionally has or extends along the second attachment axis or a third attachment axis. A third tip of the third spike component forms the third distal end. In other words, the third distal end is a third tip of the third spike component. The third base may be arranged at or constitute the third proximal end of the third attachment part. The third spike component may have a length in the range from 1 mm to 15 mm such, as in the range from 3 mm to 10 mm. Thereby sufficient penetration into the internal tissue may be provided for while at the same time reducing the risk of damaging the internal tissue. The third distal end of the third attachment part may be provided with a tip configured to penetrate a biological tissue. The third distal end of the third attachment part may be provided with a gripping part configured to grip a biological tissue.

The third spike component may have a cross-sectional diameter in the range from 0.1 mm to 5 mm, such as in the range from 0.5 mm to 2.0 mm.

The third spike component may be straight and/or curved. The third spike component may comprise a third primary section that is straight. The third spike component may comprise a third secondary section, e.g. between the third primary section and the third distal end or between the third base and the third primary section. The third secondary section may be curved.

In one or more exemplary drug delivery devices, the third attachment part is rotationally attached to the first body part. For example, the third attachment part may be located next to the first attachment part. The third distal end may be directed in the same direction as the first distal end. The third distal end may be directed in the opposite direction as the first distal end. The third distal end may be directed at an angle with respect to the first distal end. The third attachment part may be on the same first rotation axis as the first attachment part. The third attachment part may have a third rotation axis, such as via a third joint connection.

In one or more exemplary drug delivery devices, the third attachment part is rotationally attached to the second body part or the first body part. For example, the third attachment part may be located next to the second attachment part or the first attachment part. The third distal end may be directed in the same direction as the second distal end. The third distal end may be directed in the opposite direction as the second distal end. The third distal end may be directed at an angle with respect to the second distal end. The third attachment part may be on the same first rotation axis as the second attachment part. The third attachment part may have a third rotation axis, such as via a third joint connection.

In one or more exemplary drug delivery devices, the third attachment part is rotationally attached to the second body part. For example, the third attachment part may be located next to the second attachment part. The third distal end may be directed in the same direction as the second distal end. The third distal end may be directed in the opposite direction as the second distal end. The third distal end may be directed at an angle with respect to the second distal end. The third attachment part may have a third rotation axis, such as via a third joint connection. The third rotation axis may be parallel to, or in line with, the second rotation axis.

In one or more exemplary drug delivery devices, the third attachment part is optionally configured to rotate about a third rotation axis. The third rotation axis may be parallel to the central axis and/or the primary axis. The third rotation axis may form a third angle with the central axis and/or the primary axis. The third angle may be less than 15°. The second angle may be in the range from 75° to 105°, such as 90°±5° or 90°.

In one or more exemplary drug delivery devices, the third attachment part extends, e.g. at least in an activated state of the drug delivery device and optionally in an initial state of the drug delivery device, in a direction optionally away from the drug delivery device. In other words, the third attachment part may extend, e.g. at least in an activated state of the drug delivery device and optionally in an initial state, from an outer surface of the drug delivery device. Formulated differently, the third attachment axis may, e.g. at least in an activated state of the drug delivery device and optionally in an initial state of the drug delivery device form an angle of at least 45° with the central axis and/or the primary axis.

The third attachment part may in a first state of the drug delivery device extend in a third primary direction and in a third state of the drug delivery device extend in a third secondary direction. The third primary direction and the third secondary direction may form an angle of at least 30°. The third primary direction may be parallel or substantially parallel to the central axis. The third primary direction may form an angle less than 60° with the central axis. The third secondary direction may form an angle of at least 60°, such as about 90° with the central axis. The third secondary direction may be perpendicular to the central axis.

The third distal end of the third attachment part may be configured to move or be moved from a third primary position in a first state of the drug delivery device to a third secondary position in the second state.

In one or more exemplary drug delivery devices, further (e.g., multiple) attachment parts may be utilized. They may be rotatably attached to the first body part and/or the second body part. The multiple further attachment parts may include the same components as discussed above with respect to the first attachment part, and/or the second attachment part, and/or the third attachment part.

In one or more exemplary drug delivery systems, the drug delivery device can further include a cover element (e.g., cover, shield, protector, lock). The drug delivery device can include a plurality of cover elements.

In one or more exemplary drug delivery devices, the first cover band could be in the shape of a capsule part. For example, the first cover band could form a first half of a capsule. The first cover band could form a first half of a capsule and a second cover band could form a second half of the capsule. When fitted together, the first cover band and the second cover band could form a full capsule.

The cover element may form of a cover element body (e.g., partial cylinder, band, ring, loop, partial ring, partial loop). The cover element body may have a circumferential length greater than a longitudinal width. For example, the circumferential length may be 2×, 3×, 4×, 5×, 6×, 7×, 8×, 9×, or 10× the longitudinal width.

In one or more exemplary drug delivery devices, the cover element may fit on an outer surface of the drug delivery device and/or the carrier. The cover element may fit on an outer surface of the first body part. The cover element may fit on an outer surface of the second body part. A first cover element may fit on the outer surface of the first body part and a second cover element may fit on the outer surface of the second body part. In one or more exemplary drug delivery devices, a plurality of cover elements may be fit on a first body part and/or a second body part. For example, the cover element may be snap fit onto an outer surface of the drug delivery device. The cover element may include a cover attachment element configured to fit within a mating recess of the drug delivery device.

The cover element may partially or fully cover the first recess. The cover element may partially or fully cover the second recess. A first cover element may partially or fully cover the first recess and a second cover element may partially or fully cover the second recess. Thus, the cover element may prevent motion of the first attachment part and/or the second attachment part.

The cover element may extend fully along an outer circumference of the drug delivery device. The cover element may extend around 10, 15, 20, 25, 30, 35, 40, 45, 50, 55, 60, 65, 70, 75, 80, 85, 90, 95, or 100% of an outer circumference of the drug delivery device. The cover element may extend around greater than 10, 15, 20, 25, 30, 35, 40, 45, 50, 55, 60, 65, 70, 75, 80, 85, 90, or 95% of an outer circumference of the drug delivery device. The cover element may extend around less than 10, 15, 20, 25, 30, 35, 40, 45, 50, 55, 60, 65, 70, 75, 80, 85, 90, 95, or 100% of an outer circumference of the drug delivery device.

In one or more exemplary drug delivery devices, the cover element can also be a locking mechanism. For example, the cover element can be used to lock the first body part with relation to the second body part. The cover element may not be used as a locking mechanism.

In one or more exemplary drug delivery devices, the cover element may include one or more mating protrusions (e.g., cover element protrusions, extensions, tabs, fingers, projections, teeth). For example, the cover element may include 1, 2, 3, 4, 5, 6, 7, 8, 9, or 10 mating protrusions. The cover element may only extend longitudinally from one side of the cover element. The mating protrusions may extend longitudinally from both sides of the cover element. The mating protrusions may be equally spaced along the cover element. The mating protrusions may be unequally spaced along the cover element. In one or more exemplary drug delivery devices, the cover element may include at least one mating protrusion configured to mate with a slot on the first body part and/or the second body part.

The one or more mating protrusions may extend towards a longitudinal center of the drug delivery device (e.g., along an outer surface of the body towards the second body part if the cover element is located on the first body part, or along an outer surface of the body towards the first body part if the cover element is located on the second body part).

The one or more mating protrusions may be triangular, square, rectangular, rounded, or other polygonal shapes. The one or more mating protrusions may vary in shape along the cover element.

In one or more exemplary drug delivery devices, the drug delivery device may include mating features. The mating features can be configured to mate with the one or more protrusions of the cover element. The mating features can include one or more body mating protrusions (e.g., extensions, tabs, fingers, projections, teeth) extending radially outward from an outer surface of the drug delivery device. The mating features can extend from the first body part, the second body part, or both. The mating features can be formed in one or more circumferential rows. For example, there can be one circumferential row of mating features or two circumferential rows of mating features. Both circumferential rows can be on the same body part (e.g., the first body part or the second body part). In alternative implementations, one circumferential row of mating features can be on the first body part and a second circumferential row of mating features can be on the second body part.

The one or more mating protrusions may be triangular, square, rectangular, rounded, or other polygonal shapes. The one or more mating protrusions may vary in shape along the first locking band. The one or more mating protrusions may be angled in a circumferential direction to form a mating recess (e.g., curve, cavity, space, gap). This mating recess can help lock the one or more mating protrusions to the one or more protrusions of the first locking band. Further, the mating recess can prevent unwanted release of the cover element.

Accordingly, when the cover element is attached to the drug delivery device, the one or more mating protrusions can fit between the one or more body mating protrusions. The one or more mating protrusions can fit within adjacent body mating protrusions. If used as a locking mechanism, this can prevent rotation of the first body with respect to the second body part.

For example, the first body will be impeded in rotating by the cover element. In some embodiments, the mating protrusion can be located between two mating protrusions, each of the mating protrusions angled in opposite directions to hold the locking protrusion in place. If not used as a locking mechanism, the mating protrusions can help properly fit the cover element on the drug delivery device.

In some implementations the mating features may be recesses extending internally into the drug delivery device. The mating protrusions can then extend radially inward instead of longitudinally to mate with the mating features.

As discussed above, if used as a locking mechanism, when the cover element is attached and the one or more mating protrusions mate with the mating features, the first body part is locked in place with relation to the second body part. Specifically, the cover element may be configured to rotationally lock the first body part in relation to the second body part, or vice versa. The first body part and the second body part may be released from the cover element when the cover element dissolves as discussed herein.

Regardless of whether the cover element is used as a locking mechanism or not, the dissolving of the cover element can allow the first attachment part and/or the second attachment part, depending on the coverage of the cover element, to further rotate out of the respective first or second recess. Thus, when the first body and the second body rotate with respect to one another, the first attachment part and the second attachment part can rotate for inserting into tissue.

As mentioned, the drug delivery device comprises an actuator mechanism. The actuator mechanism is configured to move the first attachment part in relation to the second attachment part, such as configured to move the first distal end towards and/or away from the second distal end, e.g., at least during a part of a rotation, such as in a first rotation and optionally in a second rotation. In one or more exemplary delivery devices, the actuator mechanism is configured to move the first distal end towards the second distal end. To move the first distal end towards the second distal end may be understood as reducing a distance between the first distal end and the second distal end. To move the first distal end towards the second distal end may be understood as reducing an angle between the first attachment axis and the second attachment axis, such as reducing an angle between a first secondary direction of the first attachment part and a second secondary direction of the second attachment part. In one or more exemplary drug delivery devices, the actuator mechanism reduces the distance between the first distal end and the second distal end during rotation of the first body part with respect to the second body part around the central axis.

In one or more exemplary drug delivery devices, the actuator mechanism is configured to move the first distal end towards the second distal end by rotating, e.g., in a second state of the drug delivery device, the first body part in relation to the second body part and/or vice versa. The actuator mechanism may be configured to rotate the first body part at least 90°, such as at least 450°, at least 810°, at least 1170°, at least 1530°, or even at least 1890° in relation to the second body part about the primary axis. The actuator mechanism may be configured to rotate the first body part in relation to the second body part about the primary axis in a stepwise manner. In other words, to rotate the first body part in relation to the second body part about the primary axis may comprise a plurality of rotations including a first rotation and a second rotation, e.g. a first rotation followed by a first time period with reduced or no rotation followed by a second rotation. A first rotation followed by a second rotation after a first time period may increase the possibility of the drug delivery device attaching to the biological tissue. The first time period or in general time periods between rotations allows the drug delivery to move to other positions in the gastrointestinal tract. In other words, if the drug delivery does not attach to the biological tissue during a first rotation, further rotations increases the chance of attachment to the internal tissue. The first rotation may be at least 90°, and the second rotation may be at least 180º. The plurality of rotations may comprise a third rotation. The third rotation may be at least 180º.

In one or more exemplary drug delivery devices, a movement of the first distal end towards the second distal end may be preceded by and/or followed by a movement of the first distal end away from the second distal end. In other words, a movement of the first distal end towards the second distal end may be prior to and/or after movement of the first distal end away from the second distal end. For example, a first rotation may comprise moving the first distal end towards the second distal end and/or moving the first distal end away from the second distal end. For example, a second rotation may comprise moving the first distal end towards the second distal end and/or moving the first distal end away from the second distal end. For example, a third rotation may comprise moving the first distal end towards the second distal end and/or moving the first distal end away from the second distal end.

The actuator mechanism optionally comprises a resilient part such as a spring element configured to apply force to the first body part and/or the second body part. The resilient part may comprise a first part, such as a first end, connected to the first body part. The resilient part may comprise a second part, such as a second end, connected to the second body part.

In one or more exemplary drug delivery devices, the actuator mechanism optionally comprises a swelling media, i.e., a media increasing its volume, e.g., upon contact with a fluid, e.g., in order to provide rotation of parts in relation to each other. In one or more exemplary drug delivery devices, a swelling medial provides rotation of the first attachment part in relation to the first body part and/or provides rotation of the second attachment part in relation to the second body part. In one or more exemplary drug delivery devices, a swelling medial provides rotation of the first body part in relation to the second body part.

The actuator mechanism, such as the resilient part, may be configured to rotate the first attachment part about the first rotation axis in relation to the first body part.

The actuator mechanism, such as the resilient part, may be configured to rotate the second attachment part about the second rotation axis in relation to the second body part.

In one or more exemplary drug delivery devices, the first attachment part and the second attachment part form an angle when the first distal end and the second distal end are in a plane that includes the primary axis. In other words, the first attachment axis and the second attachment may form an angle, e.g., larger than 5°, such as in the range from 10° to 75°, when the first distal end and the second distal end are in a plane that includes the primary axis. In one or more exemplary drug delivery devices, the first attachment part and the second attachment part comprise a first spike component and a second spike component, respectively.

In one or more exemplary drug delivery devices, the drug delivery device has a first state, also denoted initial state, where the first body part and the second body part are rotationally stationary relative to each other and a second state, also denoted activated state, where the first body part and the second body part are rotationally mobile relative to each other, e.g., can rotate about the primary axis of the drug delivery device. In other words, the first body part may be locked, e.g., prevented from rotating, in relation to the second body part. The first state may e.g., be an initial state or introduction state, where the drug delivery device is adapted to be introduced into the body, and where the first body part and the second body part are stationary relative to each other. In the first state the resilient part may have a predefined amount of stored energy, where the energy level is stationary in the resilient part while the body parts are stationary.

In one or more exemplary drug delivery devices, the drug delivery device has a first state where the resilient part has a constant resilient force load and a second state where the resilient part at least partly releases the resilient force load. In other words, the resilient part may be biased or preloaded in the first state of the drug delivery and upon release, e.g., by release of a locking mechanism, (i.e., the drug delivery device being in the second state) the force from the resilient part may affect a rotation of the first body part in relation to the second body part, i.e., including a movement of the first distal end towards the second distal.

In one or more exemplary drug delivery devices, the actuator mechanism is configured to move the first distal end from a first primary position, e.g. in first state of the drug delivery device, with a first primary radial distance from a central axis of the delivery device to a first secondary position, e.g. in second state of the drug delivery device, with a first secondary radial distance from the central axis and/or primary axis, wherein the first secondary radial distance is larger than the first primary radial distance. Thus, the first distal end of the first attachment may be in a first primary position when the drug delivery device is in the first state and/or the first distal end of the first attachment part may be in a first secondary position when the drug delivery device is in the second state.

The first primary radial distance may be less than 10 mm, such as less than 8 mm or even less than 5 mm. The first secondary radial distance may be larger than the first primary radial distance. The first secondary radial distance may be larger than 5 mm, such as larger than 6 mm, or larger than 8 mm. In one or more exemplary drug delivery devices, the first secondary radial distance is in the range from 6 mm to 15 mm.

In one or more exemplary drug delivery devices, the first attachment part, such as a part of the first spike component and/or the first distal end, may, in the first state be arranged or at least partly arranged within a first primary recess of the first body part. In the first state, the first distal end may be arranged inside the first body part.

In one or more exemplary drug delivery devices, the first attachment part, such as a part of the first spike component and/or the first distal end, may, in the second state be arranged or at least partly arranged outside the first primary recess of the first body part.

In one or more exemplary drug delivery devices, the first attachment part, such as a part of the first spike component and/or the first distal end, may, in the first state be arranged within a second primary recess of the second body part. Thereby, the first attachment part may be configured to lock the first body part in relation to the second body part in the first state of the drug delivery device.

In one or more exemplary drug delivery devices, the first attachment part, such as a part of the first spike component and/or the first distal end, may, in the second state be arranged outside the second body part and/or at least outside the second primary recess of the second body part.

In one or more exemplary drug delivery devices, the actuator mechanism is configured to move the second distal end from a second primary position, e.g. in first state of the drug delivery device, with a second primary radial distance from a central axis of the delivery device to a second secondary position, e.g. in second state of the drug delivery device, with a second secondary radial distance from the central axis and/or primary axis, wherein the second secondary radial distance is larger than the second primary radial distance. Thus, the second distal end of the second attachment may be in a second primary position when the drug delivery device is in the first state and/or the second distal end of the second attachment part may be in a second secondary position when the drug delivery device is in the second state.

The second primary radial distance may be less than 10 mm, such as less than 8 mm or even less than 5 mm. The second secondary radial distance may be larger than the second primary radial distance. The second secondary radial distance may be larger than 5 mm, such as larger than 6 mm, or larger than 8 mm. In one or more exemplary drug delivery devices, the second secondary radial distance is in the range from 6 mm to 15 mm.

In one or more exemplary drug delivery devices, the second attachment part, such as a part of the second spike component and/or the second distal end, may, in the first state be arranged or at least partly arranged within a first secondary recess of the first body part. Thereby, the second attachment part may be configured to lock the first body part in relation to the second body part in the first state of the drug delivery device. In the first state, the second distal end may be arranged inside the second body part.

In one or more exemplary drug delivery devices, the second attachment part, such as a part of the second spike component and/or the second distal end, may, in the second state be arranged or at least partly arranged outside the first body part and/or at least outside the first secondary recess of the first body part.

In one or more exemplary drug delivery devices, the second attachment part, such as a part of the second spike component and/or the second distal end, may, in the first state be arranged within a second secondary recess of the second body part.

In one or more exemplary drug delivery devices, the second attachment part, such as a part of the second spike component and/or the second distal end, may, in the second state be arranged outside the second recess of the second body part.

In one or more exemplary drug delivery devices, the actuator mechanism is configured to move, e.g., by rotation about a first rotation axis of the first attachment part (first base part), the first distal end from a first primary angular position of first primary position to a first secondary angular position of first secondary position in relation to a first proximal end of the first attachment part. An angle between the first primary angular position and the first secondary angular position may be larger than 10°, such as larger than 45° or larger than 60°.

In one or more exemplary drug delivery devices, the actuator mechanism is configured to move, e.g., by rotation about a second rotation axis of the second attachment part (second base part), the second distal end from a second primary angular position of second primary position to a second secondary angular position of second secondary position in relation to a second proximal end of the second attachment part. An angle between the second primary angular position and the second secondary angular position may be larger than 10°, such as larger than 45° or larger than 60°.

In one or more exemplary drug delivery devices, the drug delivery device comprises a locking mechanism. The locking mechanism may be the cover element as discussed above. Alternatively, the locking mechanism be different from the cover element. Thus, both a cover element and a locking mechanism can be used. In one or more exemplary embodiments, the cover element can act as the locking element discussed above and the locking mechanism can be used as well. In one or more exemplary embodiments, the cover element does not act as a locking element and the locking mechanism can be the only locking element.

The locking mechanism may be configured to lock, e.g., prevent rotation of the first body part in relation to the second body part in a first state of the drug delivery device. The locking mechanism may be configured to lock the first attachment part in a first primary position, e.g., in relation to the first body part, when the drug delivery device is in the first state. Upon release of the locking mechanism, the first attachment part may be allowed to move from a first primary position to a first secondary position. The locking mechanism may upon release be configured to allow rotation of the first body part in relation to the second body part, e.g., in a second state of the drug delivery device. The locking mechanism may be configured to lock the second attachment part in a second primary position when the drug delivery device is in the first state. The locking mechanism may upon release be configured to allow the second attachment part to move from a second primary position to a second secondary position.

The locking mechanism may comprise a first locking element optionally configured to lock and/or unlock (release) the first body part in relation to the second body part. The first locking element may be configured to lock and/or unlock (release) the first attachment part in relation to the first body part. The first locking element may be configured to lock and/or unlock (release) the second attachment part in relation to the second body part. The first locking element may be arranged in a first primary recess of the first body part and/or in a second primary recess of the first body part and/or the second body part. The first locking element may be configured to dissolve when the drug delivery device enters the gastrointestinal tract or at a desired location in the gastrointestinal tract, thereby releasing the first body part in relation to the second body part and allowing the actuator mechanism to rotate the first body part in relation to the second body part and thereby moving the first distal end towards the second distal end in turn resulting in attachment of the drug delivery device to the internal tissue.

The locking mechanism may comprise a second locking element optionally configured to lock and/or unlock (release) the first body part in relation to the second body part. The second locking element may be configured to lock and/or unlock (release) the second attachment part in relation to the second body part. The second locking element may be arranged in a first secondary recess of the first body part and/or in a second secondary recess of the first body part and/or the second body part. The second locking element may be configured to dissolve when the drug delivery device enters the gastrointestinal tract, thereby unlocking or releasing the first body part in relation to the second body part and allowing the actuator mechanism to rotate the first body part in relation to the second body part and thereby moving the first distal end towards the second distal end in turn resulting in attachment of the drug delivery device to the internal tissue.

The material and/or properties of the cover element and/or the first locking element and/or the second locking element and/or the sleeve and/or the coupling component and/or the first spike component may be selected such that the release of the body parts and/or activation of the drug delivery is controlled to take place at a desired location in the gastrointestinal tract, such as in the stomach or in the intestines. The material of the cover element and/or the first locking element and/or the second locking element and/or the sleeve and/or the coupling component and/or the first spike component may comprise one or more of sugars, sugar derivatives, hydrophilic polymers, pH dependent polymers, and pharmaceutically acceptable excipients that disperses, dissolves, swells, and/or gels upon contact with water/fluid.

In one or more exemplary drug delivery devices, at least part of the first attachment part and/or the second attachment part may be made of a biodegradable material, absorbable material, or similar material which allows the material of the attachment part to be broken down, degraded and/or dissolved by processes that are present in the body, such as corrosion, degradation, hydrolysis and/or proteolytic enzymatic degradation. Thus, when the attachment part(s) has been inside the human body for a period of time, the attachment part(s) may dissolve, decompose or degrade to such a degree that the attachment part may lose its structural stability, which may in turn release the drug delivery device from the surface it has attached itself to. Thus, after a period, e.g., when the drug substance has been released from the attachment part(s), the attachment part(s) may deteriorate to such a degree that the drug delivery device may be released and may continue its journey through the gastrointestinal tract to be released through natural intestinal and/or bowel movements of the user or patient.

In one or more exemplary drug delivery devices, the rotational axis of the first body part and/or the second body part (primary axis) may be the central axis of the drug delivery device, e.g., the primary axis of the first body part may be coaxial to the central axis. Thus, the central axis intersects both the first body part and the second body part, and may define the primary axis.

In one or more exemplary drug delivery devices, the first body part and the second body part may be substantially symmetrical in a radial direction perpendicular to the central axis. This may mean that the first body part and/or the second body part may have a circular periphery, where the periphery may extend in a radial direction away from and perpendicular to the central axis.

The first attachment axis may be seen as an axis that is coaxial with the length of the first attachment part. The second attachment axis may be seen as an axis that is coaxial with the length of the second attachment part. In case the first attachment part has a shape that is not straight, the first attachment axis may be defined as an axis that intersects the first distal end and the first proximal end of the first attachment part. In case the second attachment part has a shape that is not straight, the second attachment axis may be defined as an axis that intersects the second distal end and the second proximal end of the second attachment part.

In one or more exemplary drug delivery devices, the first attachment axis may be positioned at a first distance from the central axis, while the second attachment axis may be positioned at a second distance from the central axis and/or primary axis.

For example, the first attachment axis may be positioned at a first primary distance from the central axis in the first state of the drug delivery device. The first primary distance may be larger than 0.5 mm, such as in the range from 1 mm to 15 mm or larger than 1 mm, e.g., 2 mm, 3 mm, 4 mm, 5 mm, 6 mm, 7 mm, 8 mm, 9 mm, 10 mm, 11 mm, 12 mm, 13 mm, or 14 mm.

The first attachment axis may cross or be close to (distance less than 0.5 mm) the central axis in the first state of the drug delivery device.

The first attachment axis may be positioned at a first secondary distance from the central axis in the second state of the drug delivery device. The first secondary distance may be larger than 0.5 mm, such as in the range from 1 mm to 15 mm or larger than 1 mm, e.g., 2 mm, 3 mm, 4 mm, 5 mm, 6 mm, 7 mm, 8 mm, 9 mm, 10 mm, 11 mm, 12 mm, 13 mm, or 14 mm.

The first attachment axis may cross or be close to (distance less than 0.5 mm) the central axis in the second state of the drug delivery device.

For example, the second attachment axis may be positioned at a second primary distance from the central axis in the first state of the drug delivery device. The second primary distance may be larger than 0.5 mm, such as in the range from 1 mm to 15 mm or larger than 1 mm, e.g., 2 mm, 3 mm, 4 mm, 5 mm, 6 mm, 7 mm, 8 mm, 9 mm, 10 mm, 11 mm, 12 mm, 13 mm, or 14 mm.

The second attachment axis may cross or be close to (distance less than 0.5 mm) the central axis in the first state of the drug delivery device.

The second attachment axis may be positioned at a second secondary distance from the central axis in the second state of the drug delivery device. The second secondary distance may be larger than 0.5 mm, such as in the range from 1 mm to 15 mm or larger than 1 mm, e.g., 2 mm, 3 mm, 4 mm, 5 mm, 6 mm, 7 mm, 8 mm, 9 mm, 10 mm, 11 mm, 12 mm, 13 mm, or 14 mm.

The second attachment axis may cross or be close to (distance less than 0.5 mm) the central axis in the second state of the drug delivery device.

In one or more exemplary drug delivery devices, the first attachment part (first attachment axis) and/or the second attachment part (second attachment axis) may be configured to be at an angle to each other when they intersect a plane that includes the central axis. The plane may be a plane that contains the central axis, where the plane also includes a radial axis extending at a right angle from the central axis. When the first attachment part intersects the plane, the first attachment axis of the first attachment part may be at an angle to the plane so that the first distal end of the first attachment part is the first part of the first attachment part that intersects the plane, where the remaining parts of the first attachment part intersects the plane subsequently during rotational movement. The second attachment part may intersect the same plane from the opposite side, where the second distal end of the second attachment part is the first part of the second attachment part that intersects the plane, where the remaining parts of the second attachment part intersects the plane subsequently during rotational movement. Thus, the second attachment part optionally intersects the plane from an opposing rotational direction than the first attachment part. This may also mean that when the first distal end and the second distal end of respective first attachment part and second attachment parts each contact the plane, the first attachment part (first attachment axis) is at an angle to the plane as well as at an angle to the second attachment part (second attachment axis). The angle between the first attachment part (first attachment axis) and the second attachment part (second attachment axis) to the plane may be approximately half the size of the angle between the first attachment part and the second attachment part.

In one or more exemplary drug delivery devices, the first body part may be configured to rotate in a first direction and the second body part may be configured rotate in a second direction, where the first direction is opposite to the second direction. Thus, as an example, the first body part may rotate in a clockwise direction, while the second body part may rotate in an opposed anti clockwise direction. In one or more examples where the drug delivery device comprises three or more body parts, abutting or neighbouring body parts may rotate in opposite directions. This may also mean that every second body part may rotate in the same direction. For example, where a first body part and a third body part rotate in the same first direction, then a second body part and/or a fourth body part may rotate in a second direction opposite the first direction.

In one or more exemplary drug delivery devices, the actuator mechanism may comprise one or more resilient parts, such as a plurality of resilient parts.

In one or more exemplary drug delivery devices, the first distal end of the first attachment part and/or the second distal end of the second attachment part may be provided with a sharp tip configured to penetrate a biological tissue. The sharp tip may be positioned in the vicinity of the distal end of the respective attachment part, where the sharp tip may be configured to have a diameter at the distal end which is smaller than a diameter of the attachment part at a distance from the distal end. The sharp tip may be configured in such a manner that when a rotational force is applied to the first attachment part, and a counterforce is applied to the second attachment part, the counterforce may cause the sharp tip to penetrate the biological tissue due to the force applied by the actuator mechanism.

When the first attachment part and/or the second attachment part penetrates the biological tissue due to the rotation between the first body part and the second body part (the first distal end moving towards the second distal end, the respective penetration point(s) in the biological tissue may be utilized to deliver a drug substance from the drug delivery device into the biological tissue, and where the drug substance may be introduced into the biological tissue that is beyond the mucous membrane. Thereby, the drug substance may enter the bloodstream more easily than if the drug substance is released in the stomach or intestinal lumen, and the drug delivery may be more effective. An example of this is when the drug substance is insulin, where insulin may degrade inside the gastrointestinal tract and is not capable of being absorbed from the gastrointestinal tract, but where a mucous membrane has been penetrated, and the insulin released through the penetrated gastrointestinal wall, the insulin will remain intact and reach the bloodstream of the user via the blood vessels in the intestinal layer beyond the mucous membrane (surface).

In one or more exemplary drug delivery devices, the first attachment part and/or the second attachment part may be provided with a gripping part configured to grip a biological tissue. The gripping part may be utilized to improve traction between the attachment part and a mucous membrane, allowing the attachment part to anchor the drug delivery device inside the body of the user. The gripping part may be a part that increases a mechanical friction between the attachment part and the surface to be attached to, where the gripping part may e.g., have a hook shape, or e.g., a shape where the gripping part of the first attachment part faces the gripping part of the second attachment part, so that the biological tissue which is positioned between the first attachment part and the second attachment part is gripped between the two gripping parts.

In one or more exemplary drug delivery devices, a part of the resilient part may be connected to the first body part and a second part of the resilient part may be connected to the second body part. This means that the resilient part may be utilized to store energy, such as rotational energy or rotational force which is applied to the first body part and the second body part, where the energy is stored in the resilient part. Furthermore, when the energy is released, e.g., when a locking element is dissolved or degraded, the force may be released to both the first body part and the second body part, which in turn transfers the force to the first attachment part and the second attachment part. The resilient part may e.g., be in the form of a helical spiral spring (mainspring) and/or a spiral torsion spring, where the first body part may be wound relative to the second body part by rotating the first body part relative to the second body part. This stores energy in the mainspring by twisting the spiral tighter. The stored force of the mainspring may then rotate the first body part in the opposing direction as the mainspring unwinds. Thus, the force of the mainspring may cause the first attachment part and the second attachment parts to travel in opposing directions, and where the attachment parts may pinch the biological tissue and either pinch the tissue or penetrate the tissue in order to attach the drug delivery device to the biological tissue.

When the drug delivery device has entered the body, and has e.g., entered the desired part in the gastrointestinal tract, the drug delivery device may be configured to transform from the first state to the second state. The transformation may be initiated by different means, where e.g., the first and the second body parts may be held in the first state using a locking mechanism e.g., comprising one or more locking elements made of a dissolvable, expandable or degradable material, where the material reacts with the surroundings, such as fluids, inside the desired body part, thereby unlocking or releasing the locking mechanism. The material of the locking elements may be a material that loses its structural force when in contact with the surroundings inside the desired body part. An example may be where locking element(s) is made of a polymeric material or a sugary substance which may dissolve, expand or degrade when it comes into contact with a certain kind of fluid which may include an enzyme or a certain kind of acid inside the digestive system. When the locking element comes into contact with the reagent, the material may dissolve, expand or degrade over time, and when the rotational force of the drug delivery device exceeds the static force of the locking element, the rotational force may be released via a rotation of the first body part relative to the second body part, or vice versa.

In one or more exemplary drug delivery devices, a locking element may fix an attachment part in a position where the attachment element locks the first body part in relation to the second part, i.e., prevents the first body part from rotating in relation to the second body part. When the locking element dissolves or degrades, the attachment part can move to a secondary position where the attachment part does not lock the first body part in relation to the second part, e.g., by the actuator mechanism causing a rotation of the attachment part about a rotation axis in relation to the body part to which the attachment part is rotationally attached.

The second state of the drug delivery device may be seen as the state which is initiated by the release of energy stored in the actuator mechanism, e.g., resilient part(s) of the actuator mechanism into a rotational force of the first and/or the second body part and/or a rotational force of the first attachment part in relation to the first body part. A termination of the second state may be seen as a point in time where the energy stored in the resilient part becomes stationary again, i.e., when the attachment parts have gripped or penetrated biological tissue and/or the rotational movement between the first body part and the second body part is stopped.

In one or more exemplary drug delivery devices, the drug delivery device may have a first state where the actuator mechanism has a constant resilient force load and a second state where the actuator mechanism releases the resilient force load. In the first state, the constant resilient force load may be seen as the energy stored in the actuator mechanism, and where the resilient force load is larger than zero. The second state may be seen as a state where the actuator mechanism releases its resilient force load, where the resilient force load is reduced, e.g., approaches zero, e.g., by rotating the first body part in relation to the second body part. The second state may be terminated when the attachment parts come into contact with or penetrates a biological tissue and the resilient force load does not change, even though it has not reached zero. Thus, a third state may follow the second state, when the drug delivery device has been attached to a wall of biological material, and the resilient force load is stationary after a resilient force release.

The first attachment part and/or the second attachment part may have an unfolding function, where during the first state of the drug delivery device, i.e., the initial state of the drug delivery device, the attachment parts are positioned or arranged inside the first body part and/or the second body part, or alternatively where the first and/or second attachment parts may be folded along the sides of the body parts. Other ways of obtaining the same may be envisioned. The folded state (first state) may e.g., be maintained using a releasable locking mechanism in the form of an encapsulation similar to a drug substance capsule, a band or plug, e.g., made of gelatine, sugars or other dissolvable materials or materials that lose their structural force. Thus, the attachment parts may be held in place until the drug delivery device has entered the gastrointestinal tract, e.g., the stomach, so that the attachment parts do not interfere or damage the lining of the mouth and/or the oesophagus. Prior to or during the transition to the second state the attachment parts may extend from the body part and outwards, making the attachment parts ready to interact with a lining of the digestive system. When the attachment part or parts are in a folded or collapsed position, the distance from the central axis to the distal end of the attachment part being longer in the second state than in a first state. Thus, the diameter of the drug delivery device in the first state will be less in than the diameter of the drug delivery device in the second state.

In one or more exemplary drug delivery devices, at least part of the first attachment part and/or the second attachment part, such as the first spike component and/or the second spike component may be made of material comprising one or more of magnesium, titanium, iron and zinc which allows for accurate and precise control of the size and/or shape/geometry of the first attachment part and/or second attachment part in turn allowing for a delivery device with desired attachment capabilities and/or small production variances which is in particular important in the pharmaceutical industry.

The first attachment part, such as the first spike component, may be made of material comprising one or more of magnesium, titanium, iron and zinc. The material of the first attachment part/first spike component may be biocompatible and/or biodegradable such as a biocompatible material and/or a biodegradable material. The material of the first attachment part/first spike component may comprise one or more biodegradable polymers such as PLA and/or POLGA. Some of, part of, most of, substantially all, or all of the material of the first attachment part/first spike component may be biocompatible and/or biodegradable. The material of the first attachment part, such as the first spike component, may comprise, consist of, or essentially consist of, biocompatible and/or biodegradable material such as biocompatible and/or biodegradable metals. The material of the first attachment part, such as the first spike component, may comprise a biodegradable or bioresorbable metal or metal alloy, such as magnesium, zinc, and/or iron or an alloy comprising one or more of magnesium, zinc and iron. A biodegradable or bioresorbable metal or metal alloy may be understood as a metal or metal alloy that degrades safely within e.g. a human body in a practical amount of time, for example related to their application. The material of the first attachment part, such as the first spike component, may comprise one or more metals such as a combination of one or more metals e.g. as a metal alloy.

The second attachment part, such as the second spike component, may be made of material comprising one or more of magnesium, titanium, iron and zinc. The material of the second attachment part/second spike component may be biocompatible and/or biodegradable such as a biocompatible material and/or a biodegradable material. The material of the second attachment part/second spike component may comprise one or more biodegradable polymers such as PLA and/or POLGA. Some of, part of, most of, substantially all, or all of the material of the second attachment part/second spike component may be biocompatible and/or biodegradable. The material of the second attachment part, such as the second spike component, may comprise, consist of, or essentially consist of, biocompatible and/or biodegradable material such as biocompatible and/or biodegradable metals. The material of the second attachment part, such as the second spike component, may comprise a biodegradable or bioresorbable metal or metal alloy, such as magnesium, zinc, and/or iron or an alloy comprising one or more of magnesium, zinc and iron. A biodegradable or bioresorbable metal or metal alloy may be understood as a metal or metal alloy that degrades safely within e.g. a human body in a practical amount of time, for example related to their application. The material of the second attachment part, such as the second spike component, may comprise one or more metals such as a combination of one or more metals e.g. as a metal alloy.

An advantage of having a biodegradable material used in the attachment part(s) may be that the delivery device is able to deliver an active drug substance or payload arranged in the attachment part(s) and/or body parts of the delivery device at a specific part of the body of the subject, e.g. such as the stomach or intestines after the delivery device has attached to the internal surface, e.g. the intestinal wall, thanks to the sharp properties of the material of the attachment part(s), and for an extended period of time, since the biodegradable material will degrade gradually in time. Further, when the material of the attachment part(s) is biodegradable, the attachment part(s) will degrade in the human body and disappear after having delivered the payload/active drug substance comprised in the drug delivery device, thereby avoiding harming the human subject over time. The attachment part(s) may be configured to degrade in a period of time of hours, e.g. 2 hours, 5 hours, 10 hours, 20 hours, or 24 hours, days, e.g. 1 day, 2 days, 5 days, or weeks, e.g. 1 week, 2 weeks, 3 weeks, or 5 weeks.

The material of the attachment part(s), such as the spike(s), may comprise one or more or a combination of magnesium (Mg), zinc (Zn), and/or iron (Fe). An advantage of having the attachment part(s) of a material comprising Mg, Zn, and/or Fe may be that the shape and size of the attachment part(s) can be precisely controlled thereby providing improved attachment to the internal surface, for example to an internal wall of the intestines of the human subject.

For example, the material of the attachment part(s), such as the spike component(s), may comprise 0,001 wt. % to 100 wt % of biodegradable metal such as 0,001 wt % to 100 wt % of magnesium, 0,001 wt % to 100 wt % of zinc, 0,001 wt % to 100 wt % of iron.

The material of the attachment part(s), such as the spike component(s), may for example comprise 0,001 wt. % of Mg, 0,005 wt. % of Mg, 0.01 wt. % of Mg, 0.05 wt. % of Mg, 0.1 wt. % of Mg, 0.5 wt. % of Mg, 1 wt. % of Mg, 5 wt. % of Mg, 10 wt. % of Mg, 20 wt. % of Mg, 30 wt. % of Mg, 40 wt. % of Mg, 50 wt. % of Mg, 60 wt. % of Mg, 70 wt. % of Mg, 80 wt. % of Mg, 90 wt. % of Mg, or 100 wt. % of Mg.

The material of the attachment part(s), such as the spike component(s), may for example comprise 0,001 wt. % of Zn, 0,005 wt. % of Zn, 0.01 wt. % of Zn, 0.05 wt. % of Zn, 0.1 wt. % of Zn, 0.5 wt. % of Zn, 1 wt. % of Zn, 5 wt. % of Zn, 10 wt. % of Zn, 20 wt. % of Zn, 30 wt. % of Zn, 40 wt. % of Zn, 50 wt. % of Zn, 60 wt. % of Zn, 70 wt. % of Zn, 80 wt. % of Zn, 90 wt. % of Zn, or 100 wt. % of Zn.

The material of the attachment part(s), such as the spike component(s), may for example comprise 0,001 wt. % of Fe, 0,005 wt. % of Fe, 0.01 wt. % of Fe, 0.05 wt. % of Fe, 0.1 wt. % of Fe, 0.5 wt. % of Fe, 1 wt. % of Fe, 5 wt. % of Fe, 10 wt. % of Fe, 20 wt. % of Fe, 30 wt. % of Fe, 40 wt. % of Fe, 50 wt. % of Fe, 60 wt. % of Fe, 70 wt. % of Fe, 80 wt. % of Fe, 90 wt. % of Fe, or 100 wt. % of Fe.

The material of the attachment part(s), such as the spike component(s), may comprise a metal alloy such as Zn—Mg, Z—Fe, Mg—Fe, or Zn—Mg—Fe. The material of the attachment part(s), such as the spike component(s), may for example comprise an alloy of Zn—Mg with 0,001 wt. % of Mg, 0,005 wt. % of Mg, 0.01 wt. % of Mg, 0.05 wt. % of Mg, 0.1 wt. % of Mg, 0.5 wt. % of Mg, 1 wt. % of Mg, 5 wt. % of Mg, 10 wt. % of Mg, 20 wt. % of Mg, 30 wt. % of Mg, 40 wt. % of Mg, 50 wt. % of Mg, 60 wt. % of Mg, 70 wt. % of Mg, 80 wt. % of Mg, or 90 wt. % of Mg.

The material of the attachment part(s), such as the spike component(s), may for example comprise an alloy of Zn—Fe with 0,001 wt. % of Fe, 0,005 wt. % of Fe, 0.01 wt. % of Fe, 0.05 wt. % of Fe, 0.1 wt. % of Fe, 0.5 wt. % of Fe, 1 wt. % of Fe, 5 wt. % of Fe, 10 wt. % of Fe, 20 wt. % of Fe, 30 wt. % of Fe, 40 wt. % of Fe, 50 wt. % of Fe, 60 wt. % of Fe, 70 wt. % of Fe, 80 wt. % of Fe, or 90 wt. % of Fe.

The material of the attachment part(s), such as the spike component(s), may for example comprise an alloy of Mg—Fe with 0,001 wt. % of Fe, 0,005 wt. % of Fe, 0.01 wt. % of Fe, 0.05 wt. % of Fe, 0.1 wt. % of Fe, 0.5 wt. % of Fe, 1 wt. % of Fe, 5 wt. % of Fe, 10 wt. % of Fe, 20 wt. % of Fe, 30 wt. % of Fe, 40 wt. % of Fe, 50 wt. % of Fe, 60 wt. % of Fe, 70 wt. % of Fe, 80 wt. % of Fe, or 90 wt. % of Fe.

The material of the attachment part(s), such as the spike component(s), may for example comprise an alloy of Zn—Mg—Fe with 0,001 wt. % of Fe, 0,005 wt. % of Fe, 0.01 wt. % of Fe, 0.05 wt. % of Fe, 0.1 wt. % of Fe, 0.5 wt. % of Fe, 1 wt. % of Fe, 5 wt. % of Fe, 10 wt. % of Fe, 20 wt. % of Fe, 30 wt. % of Fe, 40 wt. % of Fe, 50 wt. % of Fe, 60 wt. % of Fe, 70 wt. % of Fe, 80 wt. % of Fe, 90 wt. % of Fe, 0,001 wt. % of Mg, 0,005 wt. % of Mg, 0.01 wt. % of Mg, 0.05 wt. % of Mg, 0.1 wt. % of Mg, 0.5 wt. % of Mg, 1 wt. % of Mg, 5 wt. % of Mg, 10 wt. % of Mg, 20 wt. % of Mg, 30 wt. % of Mg, 40 wt. % of Mg, 50 wt. % of Mg, 60 wt. % of Mg, 70 wt. % of Mg, 80 wt. % of Mg, 90 wt. % of Mg, 0,001 wt. % of Zn, 0,005 wt. % of Zn, 0.01 wt. % of Zn, 0.05 wt. % of Zn, 0.1 wt. % of Zn, 0.5 wt. % of Zn, 1 wt. % of Zn, 5 wt. % of Zn, 10 wt. % of Zn, 20 wt. % of Zn, 30 wt. % of Zn, 40 wt. % of Zn, 50 wt. % of Zn, 60 wt. % of Zn, 70 wt. % of Zn, 80 wt. % of Zn, or 90 wt. % of Zn.

The material of the spike component(s) may for example comprise an alloy comprising dysprosium, e.g. 5.0-25.5 wt % dysprosium.

The material of the spike component(s) may for example comprise an alloy comprising neodymium and/or europium, e.g. 0.01-5.0 wt % neodymium and/or europium.

The material of the spike component(s) may for example comprise an alloy comprising zinc, e.g. 0.1-3.0 wt % zinc.

The material of the spike component(s) may for example comprise an alloy comprising zirconium, e.g. 0.1-2.0 wt % zirconium.

The material of the spike component(s) may for example comprise an alloy comprising a balance of magnesium.

The material of the spike component(s) may for example comprise an alloy with 5.0-25.5 wt % dysprosium, 0.01-5.0 wt % neodymium and/or europium, 0.1-3.0 wt % zinc, 0.1-2.0 wt % zirconium, 1 ppm-0.4 wt % impurities, and a balance of magnesium.

In one or more exemplary delivery devices, the spike component(s) may partially or fully comprise RESOLOY®. In one or more exemplary delivery devices, the spike component(s) may partially or fully comprise ZK60. In one or more exemplary delivery devices, the spike component(s) may partially or fully comprise a Mg—Zn—Ca alloy.

In one or more exemplary delivery devices, the spike component(s) may be bare. In alternative delivery devices, the spike component(s) may be at least partially coated by a material. The spike component(s) may be partially coated by a material. The spike component(s) may be fully coated by a material.

In one or more exemplary delivery devices, the spike component(s) may be coated with gold (Au). For example, the gold may be sputtered onto the spike component(s). The gold may be deposited in other methods as well, and the particular method is not limiting. In one or more exemplary delivery devices, the spike component(s) may be coated with a gold alloy.

The attachment part(s), such as the spike component(s), may be made of a material comprising one or more thermoplastic or thermoset polymers. The material of the attachment part(s), such as the spike component(s), may comprise one or more active drug substances. Thus, an active drug substance may be embedded in the material of the attachment part(s), such as the spike component(s), to form a pharmaceutical composition.

In some embodiments, the attachment part(s), such as the spike component(s), may comprise for example water soluble, water insoluble, biodegradable, non-biodegradable and/or pH dependent soluble materials. In some embodiments, the attachment part(s), such as the spike component(s), may comprise a water soluble, biodegradable and/or pH-dependent material that may dissolve and/or degrade so that the attachment part(s), such as the spike component(s), lodged in the intestinal tissue may gradually degrade and/or dissolve. In some embodiments, the attachment part(s), such as the spike component(s), may comprise a water-soluble material to allow immediate release or modified release of the active drug substance depending on the material selected. In some embodiments, a water insoluble or biodegradable material may allow depot of the active drug substance in the attachment part(s), such as the spike component(s), for longer release duration (for example days, weeks or months). In some embodiments, a pH dependent soluble material may allow the attachment part(s), such as the spike component(s), to stay intact at pH conditions below the physiologic for example a pH of approximately 7.4 to remain intact in the gastrointestinal lumen, but then may dissolve once inside the gastrointestinal wall. In some embodiments, one or more water soluble, water insoluble, biodegradable and/or pH dependent materials may optionally be combined to control release of the active drug substance for example by diffusion or erosion of the attachment part(s), such as the spike component(s), for controlled release duration (for example minutes, hours, days, weeks, or months).

In some embodiments, the attachment part(s), such as the spike component(s), may be made from different compositions. For example, an outer part of the attachment part(s), such as the spike component(s), may be made of one composition and an inner core of the attachment part(s), such as the spike component(s), may be made from another composition. In some embodiments, the outer part and the inner core of the attachment part(s), such as the spike component(s), may be composed of for example a water soluble, a water insoluble, a biodegradable, and/or a pH dependent material. In some embodiments, one or more water soluble, water insoluble, biodegradable and/or pH dependent materials may be combined to control the release of the active drug substance once the attachment part(s), such as the spike component(s), may move its position from the lumen to the internal tissue for example the gastrointestinal lumen to the gastrointestinal tissue.

In some embodiments, the attachment part(s), such as the spike component(s), may be tubular and may include a tubular body and the tubular body may comprise an active drug substance for example a liquid payload comprising the active drug substance, optionally connected to a tubular attachment part so the payload with the active drug substance may flow though the attachment part(s), such as the spike component(s), into the internal tissue for example the intestinal tissue. In some embodiments, the tubular body may contain expandable such as swelling excipients that may expand by a chemical reaction for example when mixed expand in volume and/or produce a gas to advance the delivery of the payload. In some embodiments, the expansion is by osmosis.

In some embodiments, the chamber (compartment to hold active drug substance) of the first spike component may comprise a closure part for closing the chamber. The closure part may contribute to improved control of release of the active drug substance. In some embodiments, the closure part may be composed of for example a water soluble, a water insoluble, a biodegradable, and/or a pH dependent material. In some embodiments, one or more water soluble, water insoluble, biodegradable and/or pH dependent materials may be combined to control the release of the active drug substance from the chamber once the attachment part(s), such as the spike component(s), moves its position from the lumen to the internal tissue for example from the gastrointestinal lumen to the gastrointestinal tissue.

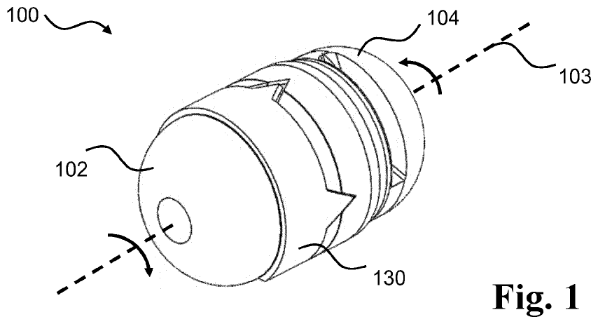

FIG. 1 shows a view of a drug delivery device in accordance with the disclosure. As shown, the drug delivery device 100 can include a first body part 102, a second body part 104, and a central axis 103 extending through the first body part 102 and the second body part 104. The first body part 102 and the second body part 104 can rotate with respect to one another around the central axis 103. The drug delivery device 100 can further include a cover element 130, as discussed further below.

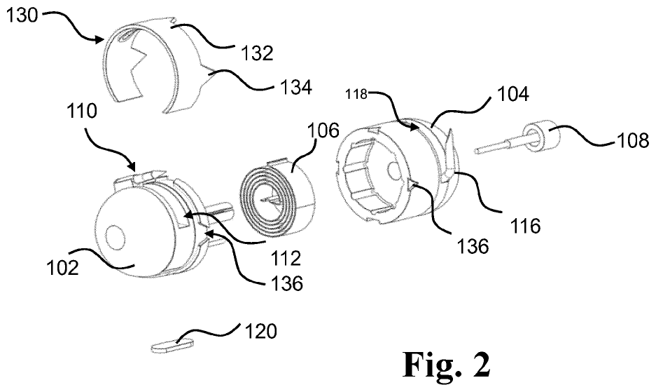

FIG. 2 shows an exploded view of the drug delivery device 100. As shown, the first body part 102 includes a first recess 112 while the second body part 104 includes a second recess 118. A first attachment part 110 can be rotatably located within the first recess 112 and a second attachment part 116 can be rotatably located within the second recess 118.

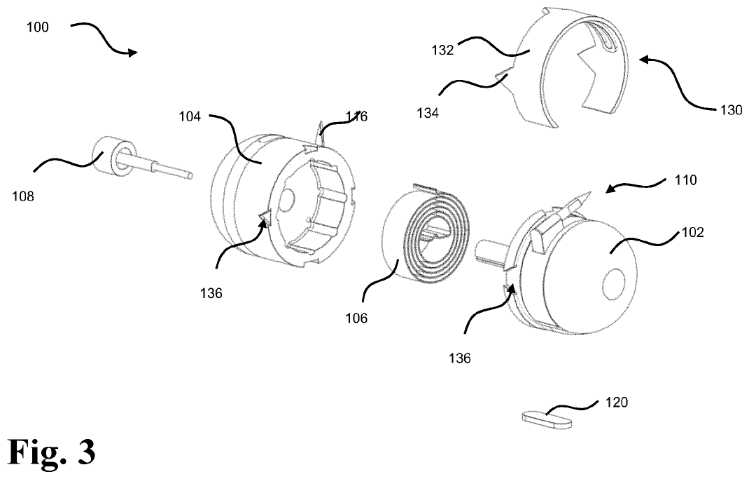

FIGS. 2-3 further show an axle 108 for rotation of the first body part 102 and the second body part 104, such as through the use of an actuator mechanism 106 for rotating the first body part 102 or the second body part 104 with respect to one another about the central axis 103. A locking mechanism 120 can prevent such rotation until the locking mechanism 120 is removed, such as by dissolving or biodegradation.

The cover element 130 can be used to cover the first recess 112. The cover element 130 could cover the second recess 118 as well, or a second cover element could be used. The cover element 130 can include a cover element body 132 with a number of mating protrusions 134 extending from the cover element body 130. The mating protrusions 134 can mate with mating features 136 of the drug delivery device 100. The mating features 136 can be located on the first body part 102 and/or the second body part 104. Thus, when the locking element 120 is removed, rotation of the first body part 102 with respect to the second body part 104 can force the cover element 130 along the central axis to uncover the first recess 112.

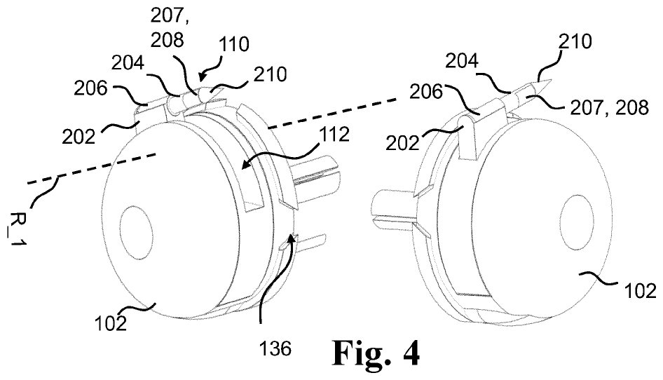

FIG. 4 shows perspective views of the first body part 102, and in particular the first attachment part 110. It is understood that the second body part 104 can include similar features. As shown, the first body part 102 can include a first attachment part 110 rotatably held within the first recess 112.

The first attachment part 110 can be made of a number of components. As shown, the first attachment part 110 may be connected with, e.g. associated with, the first body part 102 via a connector component 202. The connector component 202 can extend out of the first recess 112.

The connector component 202 can be configured to hold a first spike component 204 of the first attachment part 110 via an intermediary coupling component 206. The first spike component 204 can be releasably connected to the connector component 202. Accordingly, the coupling component 206 can be retained within the connector component 202, and the first spike component 204 can be held by the coupling component 206.

As shown, the first spike component 204 can fit within a lumen of a cylindrical coupling component 206. Specifically, the coupling component 206 can be a sleeve configured to at least partially surround the first spike component 204. In certain implementations, the sleeve can be configured to at least partially surround the connector component 202. The first spike component 204 can be made of a dissolvable material or a non-dissolvable material.

The first spike component 204 can include a first distal end 210 for penetrating tissue. The first spike component 204 may also contain an active drug substance 208, such as within chamber 207. The active drug substance 208 can be released from the chamber 207 into tissue. The chamber 207 can be exposed to an outer surface of the first spike component 204, and the coupling component 206, e.g. sleeve, is a fluid tight connection with the first spike component 204 preventing release of the active drug substance 208.

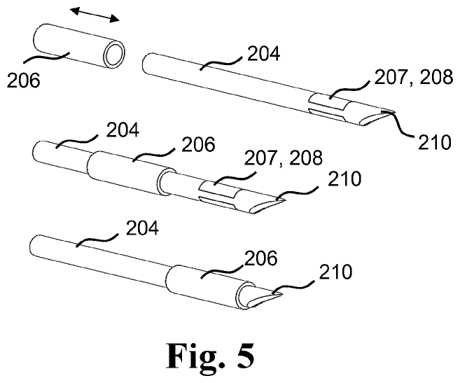

FIG. 5 illustrates an example of a first attachment element 110. As shown, the first attachment element 110 can be made of a first spike component 204 having a chamber 207 for an active drug substance 208 and a first distal end 210. The first spike component 204 can be held within a coupling component 206.

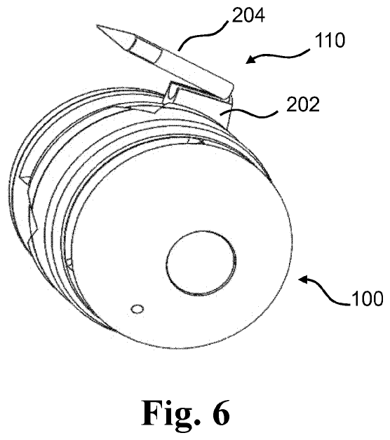

As shown, the coupling component 206 can be used to block chamber 207, thus preventing release of the active drug substance 208 from the first spike component 204. In one implementation, the coupling component 206 can dissolve, thus allowing for release of the active drug substance 208, as well as releasing the first spike component 204 from the connector component 202, and thus drug delivery device 100, as shown in FIG. 6. Alternatively, the coupling component 206 can slide with respect to the first spike component 204 to expose the chamber 207, thus allowing for release of the active drug substance 208 while retaining the connection of the first spike component 204 with the connector component 202. Alternatively, the first spike component 204 can translate completely out of the connector component 202.

Thus, the first attachment part 110 includes the coupling component 206, the coupling component 206 coupling the first spike component 204 to the connector component 202, and when the coupling component 206 decouples, the first spike component 204 is released from the connector component 202.

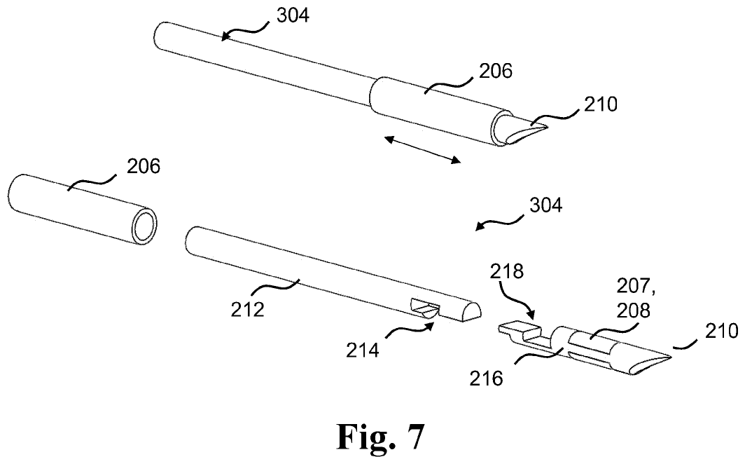

FIG. 7 illustrates an alternative first attachment element 110. As shown, the first attachment element 110 can include a first spike component 304 having a distal end 210. The first spike component 304 can be contained within a coupling component 206.

Unlike the first spike component 204 of FIG. 5, first spike component 304 can include a proximal spike component 212 having a proximal spike mating feature 214 and a distal spike component 216 having a distal spike mating feature 218. The distal spike component 216 can include the first distal end 210, as well as the chamber 207 and active drug substance 208.

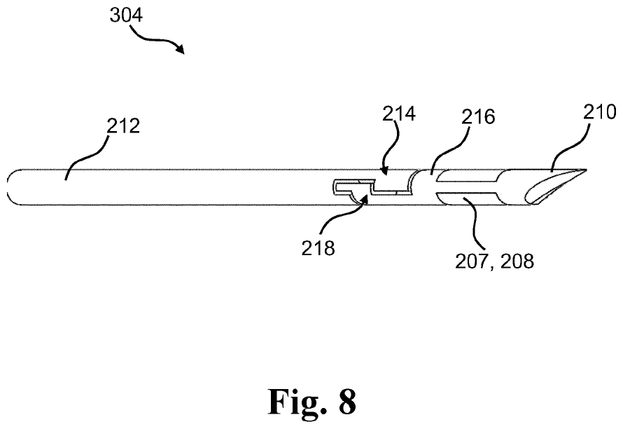

As shown in FIG. 8, the proximal spike component 212 can be attached, e.g. associated with or mated with, the distal spike component 216 via the proximal spike mating feature 214 and the distal spike mating feature 218. Further, the distal spike component 216 can be configured to separate from the proximal spike component 212 as shown in FIG. 7.

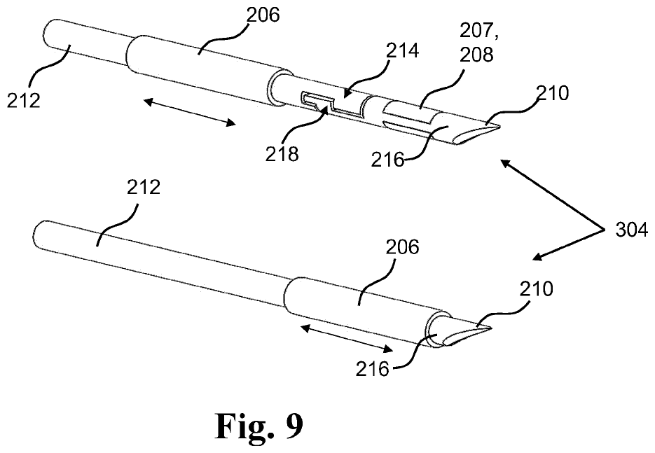

FIG. 9 illustrates the use of the coupling component 206 with respect to the first spike component 204. As shown, the coupling component 206 can translate with respect to the first spike component 304. The coupling component 206 can cover the chamber 207 and the proximal spike mating feature and distal spike mating feature 218. This prevents release of the active drug substance 208 as well as preventing release of the distal spike component 216. The coupling component 206 can be translated proximally, or the first spike component 204 translated distally, or the coupling component 206 can dissolve, to expose the proximal spike mating feature 214 and the distal spike mating feature 218. This can release the distal spike component 216, allowing it to remain in tissue with the rest of the drug delivery device 100 passing through the patient's system. As the remaining distal spike component 216 contains the chamber 207 and active drug substance 208, the active drug substance 208 can be continued to be released into the patient.

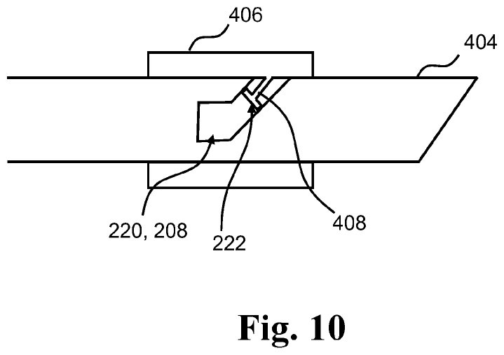

FIG. 10 illustrates an alternative first spike component 404. However, any and all of the features discussed above with respect to first spike components 204/304 can be used with this first spike component 404, and vice versa.

As shown in FIG. 10, the chamber 220, configured for holding an active drug substance 208, of the first spike component 404 can be separated from an outer surface, and is fluidly connected to the outer surface via a channel 222. The coupling component 406 can include a mating protrusion 408. The mating protrusion 408 can be inserted partially or fully within the channel 222, thus blocking fluid communication between the chamber 220 and an outer surface, e.g. blocking the channel 222. Thus prevents release of the active drug substance 208 from the first spike component 404. Once the coupling component 406 is translated away, or dissolved, the mating protrusion 408 no longer blocks the channel 222, thus allowing the active drug substance to flow from the chamber 222.

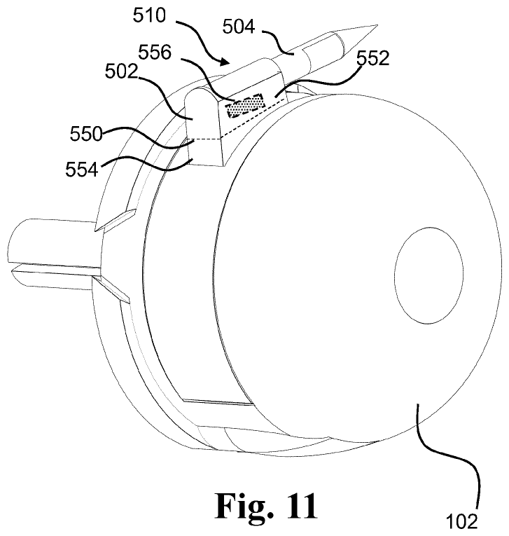

FIG. 11 illustrates a first body part 102 of a drug delivery device 100 having a connector component 502 rotatably attached to the first body part 102. The connector component 502 can be connected to a first spike component such as a first spike component 204, 304, 404 discussed in detail above.

The connector component 502 may include a pre-weakened area 550 as shown in FIG. 11. The pre-weakened area 550 can be designed to allow separation of the connector component 502 from the first body part 102. For example, as shown in FIG. 11, the pre-weakened area 550 can allow for the connector component 502 to break (e.g., separate) into a first connector section 552 and a second connector section 554. Thus, the pre-weakened area 550 can be located between the first connector section 552 and the second connector section 554. The first connector section 552 may be attached to the first spike component, such as a first spike component 204, 304, 404 discussed above, thereby releasing the first spike component from the first body part 102, and the drug delivery device 100.

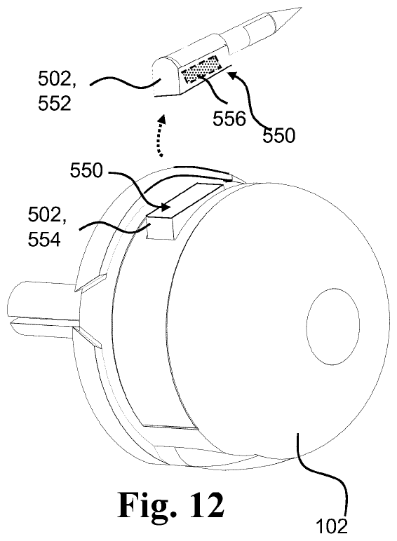

FIG. 12 illustrates the first body part 102 after separation of the first connector section 552 from the second connector section 554 along the pre-weakened area 550. Thus, the connector component 502 is configured to break into a first connector section 552 and a second connector section 554. The second connector component 554 is configured to remain attached to the first body part 102.

Alternatively, the connector component 502 can separate from the first body part 102 at an attachment point between the connector component 502 and the first body part 102. This can be, for example, at a hinge. The pre-weakened area 550 can located at an attachment point between the connector component 502 and the first body part 102. Thus, the connector component 502 may not separate into the first connector section 552 and the second connector section 554, but instead may separate from the first body part 102 as a whole.

The pre-weakened area 550 can be a biodegradable area. The pre-weakened area 550 can be a thinned area (e.g., the material may be thinner at the pre-weakened area 550). The pre-weakened area 550 can be a scored area. The pre-weakened area 550 can be a water soluble area.

The separation can occur at different times during action of the drug delivery device 100. For example, the connector component 502 can be configured to break apart from the first body part 102 during rotation of the first body part 102 and the second body part 104. Thus.

During rotation of the first body part 102 with respect to the second body part 104, the connector component 502 can separate from the first body part 102.

In other examples, the connector component 502 can be configured to break apart from the first body 102 part upon stoppage of rotation of the first body part 102 and the second body part 104. Thus, the stopping force may cause the separation of the connector component 502 from the first body part 102.

In some examples, the connector component 502 can include a high friction surface 556. The high friction surface 556 can be located on any part of the connector component 502.

For example, the high friction surface 556 can be on the first connector section 552. The high friction surface 556 can be on the second connector section 554. The high friction surface 556 can cause higher forces on the connector component 502, thus making separation from the first body part easier.

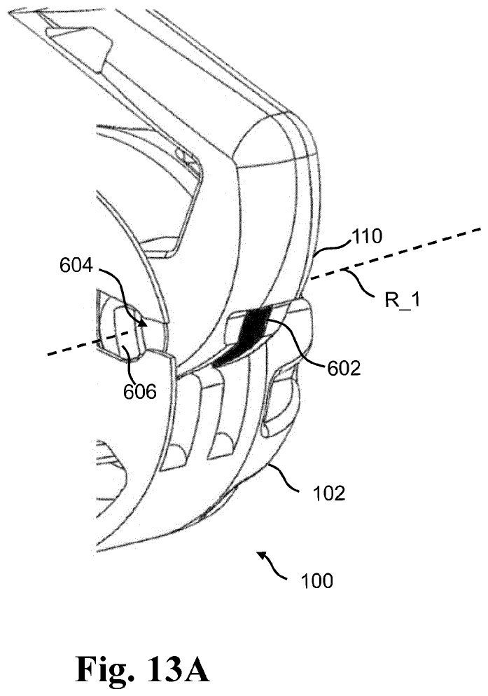

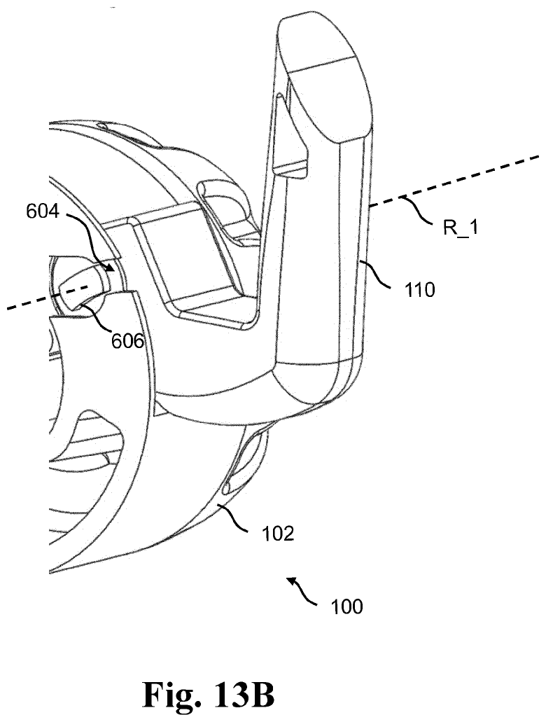

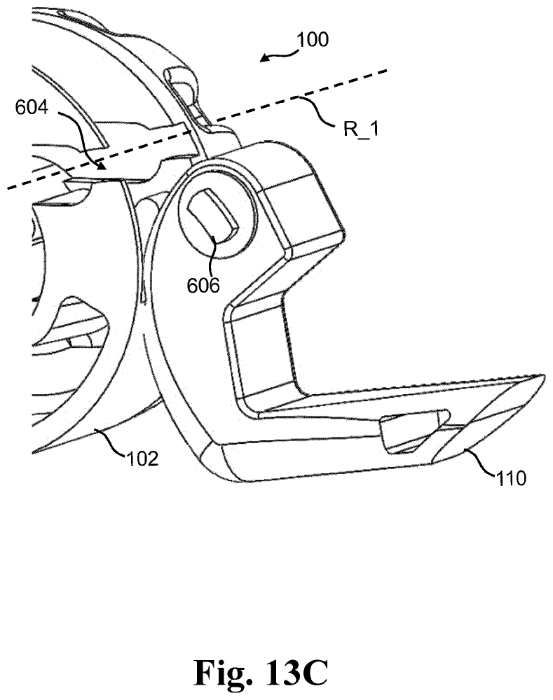

FIGS. 13A-13C show an example drug delivery device, such as including any and/or all features of the drug delivery device 100 discussed above. The drug delivery device 100 can include, for example, a first body part, e.g. including any and/or all components of first body part 102, 606 discussed above, and a first attachment part 110, e.g. including any and/or all components of the first attachment part 110 discussed above.

As shown in FIG. 13A, the first attachment part 110 includes a dissolvable component 602. The dissolvable component 602 is configured to prevent continued rotation of the first attachment part 110 with respect to the first body part 102. Further, the first body part 102 contains a slot 604, and the alignment of the first attachment part 110 prevents a protrusion 606 of the first attachment part 110 from passing or sliding through the slot 604 in the illustrated rotational position of the first attachment part 110.

FIG. 13B shows the dissolvable component 602 being dissolved, which allows the continued rotation of the first attachment part 110. Due to the alignment of the protrusion 606 with the slot 604, the protrusion 606 may be allowed to pass or slide through the slot 606. This is shown in FIG. 13C, where the first attachment part 110 is released, such as separated from, the drug delivery device 100, such as the first body part 102.

The above discussion with respect to the slot 604 and dissolvable component 602 can be used with respect to a second attachment part as well, such as second attachment part 116.

The use of the terms “first”, “second”, “third” and “fourth”, “primary”, “secondary”, “tertiary” etc. does not imply any particular order, but are included to identify individual elements. Moreover, the use of the terms “first”, “second”, “third” and “fourth”, “primary”, “secondary”, “tertiary” etc. does not denote any order or importance, but rather the terms “first”, “second”, “third” and “fourth”, “primary”, “secondary”, “tertiary” etc. are used to distinguish one element from another. Note that the words “first”, “second”, “third” and “fourth”, “primary”, “secondary”, “tertiary” etc. are used here and elsewhere for labelling purposes only and are not intended to denote any specific spatial or temporal ordering.

Furthermore, the labelling of a first element does not imply the presence of a second element and vice versa.

It is to be noted that the word “comprising” does not necessarily exclude the presence of other elements or steps than those listed.

It is to be noted that the words “a” or “an” preceding an element do not exclude the presence of a plurality of such elements.

It should further be noted that any reference signs do not limit the scope of the claims, that the exemplary embodiments may be implemented at least in part by means of both hardware and software, and that several “means”, “units” or “devices” may be represented by the same item of hardware.

Language of degree used herein, such as the terms “approximately,” “about,” “generally,” and “substantially” as used herein represent a value, amount, or characteristic close to the stated value, amount, or characteristic that still performs a desired function or achieves a desired result. For example, the terms “approximately”, “about”, “generally,” and “substantially” may refer to an amount that is within less than or equal to 10% of, within less than or equal to 5% of, within less than or equal to 1% of, within less than or equal to 0.1% of, and within less than or equal to 0.01% of the stated amount. If the stated amount is 0 (e.g., none, having no), the above recited ranges can be specific ranges, and not within a particular % of the value. For example, within less than or equal to 10 wt./vol. % of, within less than or equal to 5 wt./vol. % of, within less than or equal to 1 wt./vol. % of, within less than or equal to 0.1 wt./vol. % of, and within less than or equal to 0.01 wt./vol. % of the stated amount.

Although features have been shown and described, it will be understood that they are not intended to limit the claimed invention, and it will be made obvious to those skilled in the art that various changes and modifications may be made without departing from the spirit and scope of the claimed invention. The specification and drawings are, accordingly to be regarded in an illustrative rather than restrictive sense. The claimed invention is intended to cover all alternatives, modifications, and equivalents.

### LIST OF REFERENCES

100 drug delivery device
102 first body part
103 central axis
104 second body part
106 actuator mechanism
108 axle
110, 510 first attachment part
112 first recess
116 second attachment part
118 second recess
120 locking mechanism
130 cover element
132 cover element body
134 mating protrusion
136 mating feature
202, 502 connector component
204, 304, 404, 504 first spike component
206, 406 coupling component
207 chamber
208 active drug substance
210 first distal end
212 proximal spike component
214 proximal spike mating feature
216 distal spike component
218 distal spike mating feature
220 chamber
222 channel
408 mating protrusion
550 pre-weakened area
552 first connector section
554 second connector section
556 high friction surface
602 dissolvable component
604 slot
606 protrusion
R_1 first rotation axis

1. A drug delivery device having a central axis, the drug delivery device comprising:
a first body part;
a second body part;
an actuator mechanism configured to rotate at least one of the first body part or the second body part with respect to one another about the central axis; and
a first attachment part comprising a connector component and a first spike component, wherein the connector component is connected to the first body part and the first spike component is releasably connected to the connector component.

2-34. (canceled)

35. The drug delivery device according to claim 1, wherein the first spike component is made of a dissolvable material.

36. The drug delivery device according to claim 1, wherein the first attachment part comprises a coupling component, wherein the coupling component couples the first spike component to the connector component, and wherein when the coupling component decouples, the first spike component is released from the connector component.

37. The drug delivery device according to claim 36, wherein the coupling component comprises a sleeve configured to at least partially surround the first spike component.

38. The drug delivery device according to claim 37, wherein the sleeve is configured to at least partially surround the connector component.

39. The drug delivery device according to claim 37, wherein the sleeve is formed from a material comprising one or more of polyethylene, polypropylene, acrylonitrile butadiene styrene, polyethylene oxide, polyethylene glycol, poly(methyl methacrylate), hydroxypropylmethylcellulose acetate succinate, or hypromellose phthalate.

40. The drug delivery device according to claim 37, wherein the sleeve has a longitudinal length of 1.5 to 15 mm and a diameter of 0.15 to 2 mm.

41. The drug delivery device according to claim 1, wherein the first spike component has a chamber configured to contain an active drug substance.

42. The drug delivery device according to claim 41, wherein the first attachment part comprises a coupling component, wherein the coupling component couples the first spike component to the connector component, and wherein when the coupling component decouples, the first spike component is released from the connector component, and the coupling component comprises a sleeve configured to at least partially surround the first spike component, wherein the chamber is exposed to an outer surface of the first spike component, and wherein the sleeve provides a fluid tight connection with the first spike component preventing release of the active drug substance.

43. The drug delivery device according to claim 41, wherein the first spike component comprises a channel, which fluidly connects the chamber and an outer surface of the first spike component.

44. The drug delivery device according to claim 43, wherein the first attachment part comprises a coupling component, wherein the coupling component couples the first spike component to the connector component, and wherein when the coupling component decouples, the first spike component is released from the connector component, and the coupling component comprises a sleeve configured to at least partially surround the first spike component and, wherein the sleeve comprises a mating protrusion configured to block the channel.

45. The drug delivery device according to claim 1, wherein the connector component of the first attachment part is rotatably connected to the first body part.

46. The drug delivery device according to claim 1, wherein the first spike component comprises a distal spike component having a distal spike mating feature and a proximal spike component having a proximal spike mating feature.

47. The drug delivery device according to claim 1, wherein the coupling component is dissolvable.

48. The drug delivery device according to claim 36, wherein the first spike component is configured to translate with respect to the coupling component.

49. The drug delivery device according to claim 1, wherein the connector component is configured to separate from the first body part.

50. The drug delivery device according to claim 49, wherein the connector component is configured to separate into a first connector section and a second connector section and, wherein the second connector section is configured to remain attached to the first body part.

51. The drug delivery device according to claim 49, wherein the connector component comprises a pre-weakened area comprising a biodegradable area and/or a water-soluble area.

52. The drug delivery device according to claim 51, wherein the pre-weakened area is located between the first connector section and the second connector section or wherein the pre-weakened area is located at an attachment point between the connector component and the first body part.

53. A drug delivery device having a central axis, the drug delivery device comprising:
a first body part;
a second body part;
an actuator mechanism configured to rotate at least one of the first body part or the second body part with respect to one another about the central axis; and
a first attachment part connected to the first body part, wherein the first attachment part is configured to separate from the first body part.
